(19) United States
(12) Patent Application Publication
(10) Pub. No.: US 2010/0275278 A1
(43) Pub. Date: Oct. 28, 2010

(54) ANIMAL MODEL FOR HIV INDUCED DISEASE

(76) Inventor: Nelson M. Karp, Virginia Beach, VA (US)

Correspondence Address: WILLIAMS MULLEN, 222 CENTRAL PARK AVENUE, SUITE 1700, VIRGINIA BEACH, VA, 23462, US

(21) Appl. No.: 12/794,102

(22) Filed: Jun. 4, 2010

Related U.S. Application Data
(63) Continuation-in-part of application No. 11/702,260, filed on Feb. 5, 2007, now abandoned.
(60) Provisional application No. 60/765,315, filed on Feb. 3, 2006.

(51) Int. Cl. A01K 67/00; C12N 7/00

(52) U.S. Cl. 800/11; 435/235.1

(57) ABSTRACT

HIV does not cause disease in any non-human species. Thus, there is no animal model system to evaluate the efficacy of strategies aimed at preventing, treating or curing disease caused by this virus. The present invention provides compositions and a method for producing an animal model for HIV induced disease. The present invention is an animal adapted to simulate a human-like immune response to HIV, which is accomplished by activation and inactivation of complement of proteins within the animal. Accordingly, the present invention stages certain human proteins within an animal by way of its gut associated lymphoid tissue followed by infection of live HIV.

### RELATED U.S. APPLICATION DATA

The present application is a Continuation-in-Part of application Ser. No. 11/702,260 filed Feb. 5, 2007 which claims priority to Provisional Application No. 60/765,315, filed on Feb. 3, 2006, which are incorporated herein by reference.

### FIELD OF THE INVENTION

The present invention relates to a composition and method for producing an animal model for HIV.

### BACKGROUND OF THE INVENTION

HIV is a viral infection. Therefore, by definition, HIV is an intra cellular parasite. The virus must assimilate a variety of host cellular proteins, lipids, carbohydrates and nucleic acids into its own structure and reproductive cycle. Attempts at inoculating animals with HIV have all failed. Animals such as mice lack one or more cellular proteins or other cellular derived molecules necessary for viral replication, immune evasion and immune suppression. The purpose of this invention is to produce an animal that possesses the full complement of HIV immune mediated molecules in a manner that the animal can assimilate in trans the human derived proteins necessary for an HIV infection to proliferate. The animal will not recognize these foreign molecules as being foreign, and therefore, will not develop an immune response towards them. Furthermore, these human derived molecules will be directed towards Peyer's patches, the very site of HIV replication. The animal will be susceptible to HIV disease.

Rationale Behind an HIV Animal Model
1. Allow for an in depth and ethical study of the natural course of HIV infection. Currently all studies are on human subjects and are therefore limited by ethical considerations.
2. A testing ground for anti-retroviral drugs and other technology.
3. A testing ground for HIV based vaccines.
4. Allow the development and manufacture of effective HIV vaccines. In 1794, Edward Jenner demonstrated that inoculation of humans with extracts from cowpox lesions produce minimal systemic disease but protected the recipient from smallpox. Initially, the only way to supply the population with enough cowpox vaccine was to pass the infection (cowpox) from person to person by serial infection. This methodology, however, was complicated by transmission of other diseases such as syphilis and hepatitis and fell into disfavor. The cowpox vaccine was later passed into sheep and water buffalos in an attempt to obtain enough inoculum for the population. Recently, an unused smallpox vaccine was uncovered in New York dating back to 1876. This virus was identified as vaccinia. By 1876, the original cowpox vaccine was replaced by vaccinia virus. Vaccinia is not found in any animal studied to date. It most likely resulted as a recombination of cowpox with other pox vectors, animal and human. The U.S. Smallpox Vaccine (Dryvax by Wyeth) reserve is over thirty years old and was derived from a seed stock of a New York City Board of Health strain that was passed between twenty-two to twenty-eight times on young calves. Distribution of Dryvax ceased in 1983. Multiple retroviral vectors infect animals. Passage of HIV and one or more animal retroviruses will allow for multiple recombinant events to occur. In a manner that parallels the vaccinia vaccine derivation an HIV vaccine can be developed. Such an animal model can also be a continuous inexpensive reliable source of new fresh vaccine.

### Overview of HIV Lifecycle and Protein Requirements

A retroviral life cycle can be divided into an afferent and efferent limb. The afferent limb starts with viral attachment and ends with viral DNA integration into the host genome. The efferent limb commences with the production of viral messenger RNA and culminates with viral fission releasing immature virions.

The afferent lifecycle of the virus will be arbitrarily divided into the following steps:
1. Attachment to a target cell by its surface (SU) and transmembrane (TM) proteins. The surface protein binds to the CD4 receptor and to either the CCR5 or CXCR4 coreceptor.
2. Fusion of viral envelope and cell plasma membrane.
3. Deposition of viral core into cytoplasm.
4. Reverse transcription of viral RNA.
5. Translocation of viral pre-integration complex across nuclear membrane.
6. Integration of viral DNA into host DNA.

The efferent lifecycle of the virus will be arbitrarily divided into the following steps:
7. Transcription of viral RNA into RNA.
8. Splicing of viral RNA.
9. Translocation of early viral completely spliced RNA products (Tat, Rev and Nef) across nuclear membrane into cytoplasm.
10. Rev mediated translocation of singly spliced and unspliced viral RNA across the nuclear membrane to the cytoplasm.
11. Viral env proteins produced in cytoplasmic rough endoplasmic reticulum (RER).
12. Glycosylation and folding of env proteins in Golgi apparatus.
13. Targeting of mature envelope proteins to cytoplasmic side of plasma membrane.
14. Translation of Gag and Gag-Pol polyprotein.
15. Targeting of Gag and Gag-Pol polyprotein to host endosomal machinery.
16. Gag and Gag-Pol polyprotein cleaved by viral protease.
17. Assembly of Gag and Gag-Pol polyprotein precursors and envelope proteins at budding site.
18. Viral fission.
19. Viral maturation.

Each step delineated above relies on host derived proteins, lipids, carbohydrates and/or nucleic acids. Animals do not support the HIV lifecycle because they lack one or more necessary host derived molecules.

HIV, as with all significant viral pathogens, is able to evade the host immune response. Furthermore, HIV down regulates or deregulates the host immunologic response.

For an animal model to be successful for HIV disease, three correlates of the disease must be expressed:
1. Viral replication
2. Viral immune evasion
3. Viral immune deregulation and/or suppression

Many proteins necessary for viral replication of the host immune response are human host derived proteins that are not found in animals. These include, but are not limited to, tRNA synthetase, tRNA^{lys}, Tsg101, Tal, Staufen, LEDGF/p75, Cyclin T, CDK9 and RNA polymerase II. To create an animal model capable of not only supporting HIV replication, but also reproducing HIV disease in the animal requires the assimilation of these proteins into the animal without the animal recognizing these proteins as foreign. Success of such an animal model would rely on the lack of an immunologic response to these human proteins. Furthermore, assimilation or targeting of these proteins into the proper target tissues, predominantly Peyer's patches, the principal site of HIV replication, is necessary to reproduce an HIV infection in an alternate host.

Viral evasion of the host's immune response requires the active participation of host derived cellular proteins such as the complement control proteins CD55, CD46 and Factor H. These proteins are necessary to prevent the host's immune cells from reacting to and destroying normal tissue. By incorporating these molecules into an intact HIV virion, the virus is able to fool the immune system in a “cloak-and-dagger” method that avoids virolysis.

Immune disregulation is accomplished by the virus skewing the host towards a Th2 immune response. This is accomplished by the virus hijacking the endosomal pathway by incorporating molecules such as Tsg101, Tal and Ubiquitin. Furthermore, the viral envelope incorporates MHC-11 and CD86 molecules which are consistent with a Th2 response.

As a corollary to the above paragraph, any given protein may exhibit different and at times divergent and conflicting functions, complicating the challenge to an animal model for HIV.

### DESCRIPTION OF THE INVENTION

### Summary of the Invention

The present invention provides compositions and a method for producing an animal model for HIV induced disease. The present invention is an animal adapted to simulate a human-like immune response to HIV, which is accomplished by activation and inactivation of complement of proteins within the animal. Accordingly, the present invention stages certain human proteins within an animal by way of its gut associated lymphoid tissue followed by infection of live HIV.

### DETAILED DESCRIPTION

The present invention is directed to an animal model for HIV and the method of producing the same. Preferably, the present invention is a mouse adapted to simulate a human-like immune response to HIV, which is generated by appropriate protein behavior within the mouse. The mouse genome has been published.¹ Extensive linkage conservation/synteny between mouse and human DNA has been established.² The present invention stages certain human proteins within a mouse by way of its gut associated lymphoid tissue (GALT).

A key to protein variability lies in the primary, secondary, tertiary and quaternary structure of the protein itself. The protein may assume different secondary, tertiary and quaternary structures in various environmental conditions. Changes in ph, temperature, as well as the presence, absence, or concentration of cellular cofactors, such as calcium and magnesium, alter the structure and function of the protein. Most importantly however, proteins can be divided into basic building blocks or subunits known as motifs, each which possesses a specific function which is independent of the rest of the molecule. In some instances only a portion of the protein is directly involved in a certain metabolic process. The whole protein may or may not be needed to produce the desired effect. The subunits not directly involved in the cellular activity may affect the overall structure, stability, intracellular location and often function as a scaffold.

However it has also been demonstrated in other circumstances that a subunit of a protein that carries a significant function maintains that function when physically separated from the rest of the molecule. In such circumstances one may envision that only a portion of the protein is needed to perform the desired effect and is necessary to be encoded by recombinant DNA technology to develop an animal model for HIV. Invariant amino acids in each protein are always noted. For example, the cystine residue occupying the position of amino acid 261 of Cyclin T is absolutely required for interaction with Tat.³

The above conclusion has been demonstrated with in vitro models of human CyclinT1 (hCycT1) as it interacts with the Tat protein. A heterodimer of human CyclinT1 and Tat protein is a prerequisite to the binding of the heterodimer to the TAR sequence that initiates HIV RNA replication. The first 272 amino acids of the 726-aa hCycT1 protein are sufficient to support Tat function, TAR recognition and binding and ultimately viral replication. Even more specifically a critically defined region of hCycT1 located between residues 250 and 262 is critical for Tat and TAR binding and has been termed the Tat-TAR recognition motif (TRM).

All proteins have a characteristic half life usually measured in minutes or hours. Therefore, these proteins that support HIV replication and immune evasion need to be produced within the animal in a continuous pattern with a steady state level. The tissue concentration of the proteins supplied in trans should mirror that found in the normal human immunologic milieu.

All proteins administered to the animal model are encoded within the DNA. Recombinant technology allows introduction of human DNA into bacteria, fungi, yeast or viruses. Utilizing commensal organisms, found normally in the gut of an animal such as a mouse, rat or rabbit for this recombination the proteins of human origin necessary for HIV replication and immune evasion and immune disregulation can be introduced into the animal without the animal rejecting the proteins as foreign. The mechanisms of suppressor cells and regulatory cells found within the gut associated lymphoid tissue (GALT) prevent immunologic response to ingested food, commensal organisms and the products of the commensal organisms. Commensal organisms often produce vitamins necessary for the host to survive. Vitamins are protein based structures. By reasonable inference other proteins produced by the commensals would be assimilated into the host without an ensuing immunologic response. To replicate and survive the commensal bacteria continually produce protein and other components of its structure in excess of what is needed or incorporated into the replicating bacteria. These excess proteins do not elicit an immunologic response from the host animal.

GALT constitutes nearly 80% of the total body's immune cell population. GALT is the most comprehensive lymphoid organ system in humans. The function of GALT is a paradox and at times is in conflict with the systemic immune system. The systemic immune apparatus, under normal conditions, functions in a sterile environment devoid of pathogens and pathogen associated toxins. Therefore, any foreign matter encountered by the systemic immune system is regarded as a potentially harmful invader and the appropriate immunologic response follows. GALT, however, stands as a barrier between the human organism and an external environment replete with foreign tissue. The foreign matter includes a variety of commensal organisms, commensal derived products, pathogens, and pathogen derived products and ingested food. The entire GI tract from the mouth to the anus is functionally external to the human body. Unlike the systemic immune system, which responds vigorously to any foreign matter, GALT must differentiate between commensal organisms and their products, as well as ingested food to which an immunologic response would have adverse consequences and invading pathogens potentially lethal to the host.⁴

To affect this diversity of function, GALT is compartmentalized and, in contrast to the systemic and peripheral immune system (spleen & lymph nodes), is characterized by non-homogeneously distributed B and T cells. The phenotypic behavior, cell surface markers, developmental origins, secretory products, and hence function of the T and B cells of GALT, is markedly different from the T and B cells of the systemic system. Furthermore, GALT contains certain subsets of non-conventional lymphocytes such as γ/δ T cells. Overall GALT is characterized by afferent and efferent conduits not found in the systemic system.⁵

GALT (armed with a variety of immunologic cells not found in the systemic circulation, and patterned or clustered into characteristic vehicles not found elsewhere in the body) is capable of immunologic suppression as well as classically based Th-1 and Th-2 immune responses. Antigen uptake in GALT occurs through specialized epithelial cells known as “M” cells or “membranous” cells. Antigen uptake in GALT can also occur directly by epithelial cells in close proximity to underlying T and B cells. The uptake or assimilation of antigens through the “M” cells or epithelial cells may result in localized immune response, disseminated immune response and/or tolerance or immunosuppression. The vast majority of antigens interacting with GALT results in specific suppression of immunity for that antigenic structure. This is necessary because the primary function of GALT is to prevent an immunologic reaction to innocuous, and at times beneficial, foreign material.⁶

The final determination in GALT of immunity versus tolerance rests on many variables. These include but are not limited to the chemical structure of the antigen, the dose of the antigen administered, and the cytokine environment. Whether this phenomenon is termed suppression, anergy, deletion, ignorance, and/or immunologic deviation is irrelevant. Importantly, immunologic tolerance within GALT depends on an intact epithelial barrier.⁷

Many mechanisms have been described in the literature detailing the immune suppression observed with antigens derived from the large and small intestine. In classic immunology dendritic cells exposed to peripherally derived antigen assimilate the antigen (by a variety of mechanisms including but not limited to endocytosis, macrocytosis, pinocytosis, and cross presentation). Dendritic cells (DCs) lining the tissue have been described. The DCs then undergo a process of maturation and migrate to the most proximal lymph nodes. Expressing a “danger signal” the cells of the lymph node respond and eliminate the antigen expressed by the DCs. Recently however, DCs lining the GALT with an opposite function, one of tolerance have been described in the literature. These cells stimulate a protective immune response when stimulated by pathogens whose tropism (i.e., the phenomena observed in living organisms of moving towards each other) is confined to pathogens that infect or are confined to epithelial cells.⁸

The incorporation of the DNA encoding these human derived proteins into the commensals, herein referred to as incorporated DNA, can be done through recombinant technology with the following seven methodologies commonly used and known by those in the art.
1. Incorporation of the DNA into the bacterial, viral, yeast or fungal DNA utilizing restriction enzymes, endonucleases, exonucleases, deoxyribonucleases, ribonucleases, alkaline phosphatases, polynucleotide kinases, terminal transferases, and DNA ligases, all commercially available.⁹
2. The formation of a plasmid encoding the human protein. A plasmid is a genetic particle physically separate from the chromosomal DNA of the host cell that is stable and can function and replicate independently of the nucleus.¹⁰
3. Incorporation of the DNA into a bacteriophage. A bacteriophage is a virus with a specific affinity for bacteria and has been found in association with essentially all groups of bacteria. Like other viruses, they contain either RNA or DNA but never both.¹¹
4. Hybrid plasmid/phage vectors such as cosmids, phagemids or phasmids.¹²
5. Bacterial artificial chromosomes.¹³
6. Yeast artificial chromosomes.¹⁴
7. A combination of the above.

If incorporated into a plasmid, a promoter/regulatory region controlling the plasmid activity would need to be included. The assimilation of the protein produced by the commensal into the animal may occur by passive (ATP independent) or active (ATP dependent) means. The DNA encoding a cell penetrating peptide (CPP) may be fused with the DNA encoding the human protein(s) prior to the recombinant process incorporating the DNA into the bacteria. Many cell penetrating peptides have been defined in the literature and have been used to carry cargos (attached protein, carbohydrate or lipid molecules) into cells which would normally be impermeable to these attached structures. Cell penetrating peptides can pass through cell walls, nuclear membranes, as well as the membranes enclosing other intracellular organelles with ease.¹⁵

Alternatively, the DNA encoding the below mentioned human proteins necessary for HIV viral replication, immune evasion and immune disregulation can be spliced into the DNA of an animal. Intuitively this may seem to be the most logical answer. For some proteins such as the CD4 receptor and the CCR5 and CXCR4 co-receptor, this would be workable and perhaps preferable, since the proteins would be a component of the host cell plasma membrane. Many potential problems arise using that conceptualized framework for all the proteins. Most difficult would be the targeting of the needed proteins to the sites of HIV replication (i.e., Peyer's patches). Furthermore, encoding a protein into the DNA of an organism does not equate to transcription and translation of the DNA and protein production. 70% of the DNA in a mammal is not transcribed and has been termed “junk DNA”. Production of a transgenic or chimeric animal does not equate to tissue targeting. External control of animals genetically modified at the level of embryonic cells is problematic.

These issues may be addressed as the science relating to models progresses. However, the present invention, as a first conceptualized model, involves splicing the DNA for the needed human proteins into commensal organisms.

The host proteins necessary for HIV to attach to a target cell, penetrate the target cell and replicate within the target cell, include and are not limited to the following list. The following proteins, or the nucleotide sequences encoding these proteins, preferably should be included in a working animal model for HIV:

1. Transcription factors.
a. NF_{K} B
b. NFAT
c. Sp1

2. Cellular cofactors.
a. Cyclin T
b. CDK9/PITALRE
c. RNA polymerase II
d. Exportin 1/Crm1
e. Ran GTP
f. Ran GTPase activating protein (RanGAP)

g. Ran Binding Protein (RanBP1)

3. Cellular receptors.
a. CD4

4. Cellular coreceptors.
a. CCR5
b. CXCR4
c. CCR2B
d. CCR3
e. CCR8
f. GPR1
g. GPR15 (Bob)
h. STRL33 (Bonzo)
i. US28
j. CX3CR1 (V28)
k. APJ
l. chemR23

5. Cellular proteases.
a. Furin

6. Cellular proteins involved in the ubiquitin-proteasome pathway.
a. H-β-TrCP
b. Skp1p

7. Cellular adaptor protein.
a. AP-2

8. Human ribosomal RNA.

The host derived proteins necessary for HIV to evade the immune response include but are not limited to the following, and preferably should be included in a workable animal model for HIV. (See Table in Appendix A for a complete list of “Host Proteins Incorporated into the Intact Virus and for Pre-Integration Complex (PIC)”.

1. Plasma proteins.
a. C4 binding protein (C4b protein)
b. Factor H (Includes FHL-1, FHR1, FHR2, FHR3, FHR4, FHR5)

2. Cell membrane bound proteins.
a. Membrane cofactor protein (MCP) or CD46
b. Decay accelerating factor (CD55)
c. Complement-receptor 1 (CD35)
d. Complement-receptor 2 (CD21)
3. Homologous restriction factor (HRF).

Finally and in addition to the proteins listed above the table located in Appendix A lists the host proteins incorporated into the intact virus, the pre-integration complex (PIC) and those involved in the HIV lifecycle. It is not exhaustive as new viral protein/host protein interactions are reported in the literature with regularity. The genetic loci of the human proteins have been described in the literature and allow for restriction enzyme splicing into yeast, bacteria or plasmid DNA.

In an alternative embodiment, the activity of Human Factor H in an animal can be limited by administration of soluble complement-receptor 1 (sCR1) by adding sCR1 exogenously or by splicing the genomic sequence for sCR1 into a commensal organism. This protein binds to C3b and C4b and facilitates the breakdown of these proteins by Factor 1. By binding to C3b, sCR1 prevents complement activation by the C3 convertase. The activity of Human Factor H in thwarting the complement cascade is mimicked by sCR1.

The administration of soluble CR1 is a controlled element or variable in the animal model. sCR1 allows control of tissue levels of C3b thereby limiting the activity of the C3 and C5 convertases which mirrors the function of Factor H.

In some animal models (e.g., old world primates), and particularly cell cultures derived thereof, TRIM-α confers a potent post entry (i.e., meaning after entry into the cell) block to HIV-1 infection. Cyclophilin A (CypA) binding to viral capsid proteins results in a similar response observed in vitro for certain human cell lines. Among new world primates, only owl monkeys exhibit post-entry restriction of HIV-1 replication. More specifically, monkey kidney cells of the Aotus trivirgatus owl restrict HIV infection, but are permissive for SIV infection. HIV restriction in these cells is completely abrogated when the interaction of the HIV-1 capsid and the cellular protein CypA is disrupted. Paradoxically, the opposite is seen in human cells where capsid-CypA interaction is required for efficient intracellular HIV-1 replication. Therefore if such an animal model is used the viral capsid interaction with the host CypA protein must be severed. The use of the CypA-binding drug cyclosporine A (CsA) would be necessary if these animal models were used. Similar findings may exist in other animals but have not yet been delineated.¹⁶

The most effective weapon for immune perturbation within the HIV arsenal is the Tat protein. The Tat protein is necessary for viral replication as well. A multiplicity of immune down modulating effects of the Tat protein has been well documented in human studies. An accurate model of HIV must include Tat mediated immune suppression. This will involve the Tat protein and the host cell receptors for the Tat protein.

Expression of MHC class II genes is inhibited by the Tat protein resulting in profound immunosuppression. A central protein in class II expression is the class II trans-activator (CIITA) protein. CIITA is responsible for integrating several proteins at the promoters of MHC class II genes enhancing MHC II gene transcription and ultimately MHC II gene expression.

In human models, the Tat protein inhibits CIITA function down regulating the expression of MHC II genes. Human cyclin T1 (hCycT1) is involved in this Tat mediated immunosuppression.

In mice however, the Tat protein does not interact with the human counterpart of hCycT1, mouse cyclin T1 (mCycT1). However, the Tat protein in mice does inhibit the activity of CIITA in a mechanism that is not dependent on mCycT1. The results are the same: the down regulation of the CIITA protein, decreased MHC II production, and immunosuppression.

Co-expression of transfected human CD4, CCR5 and CXCR4 molecules into murine cell cultures allows entry of HIV-1 but replication is blocked. Murine cyclin T1 binds Tat but does not bind TAR. Transfection with human cyclin T1 restored Tat function.¹⁷

Murine cyclin T2 can bind HIV-1 Tat and facilitate TAR binding if a single residue, asparagine 260 is replaced with a cysteine residue. Interestingly, Tat from HIV-2 does bind murine cyclin T1 and murine cyclin T2. However, neither complex binds effectively the TAR residue. With both HIV-1 and HIV-2 Tat effective binding and activity of Tat on HIV replication is rescued in murine cells by the above-mentioned mutation of Cyclin T2 at amino acid number 260. Therefore, if a murine model is anticipated, mutation of Cyclin T2 at residue 260 would equate to human Cyclin T1 supplied in trans. In an alternate murine animal model, another single amino acid difference between human Cyclin T1 and murine Cyclin T1 determines species restriction of HIV-1 Tat function. In this model, replacing the tyrosine residue at amino acid 261 in the murine Cyclin T1 with a cysteine conferred effective Cyclin T1 function with Tat and TAR.¹⁸

A competent Cyclin T1 is necessary but not sufficient for HIV viral replication. This can be provided to a murine model by either one of the above-mentioned mutations in the mouse genome or by providing human Cyclin T1 in trans.

An effective block of HIV replication in a murine model is the inability of the virion to assimilate murine Factor H. HIV directly activates the classical complement pathway in rabbit, mouse and guinea pig serum. This activation results in viral neutralization by lysis.¹⁹ Factor H is bound at multiple sites to gp120 and gp41 in the intact virus.²⁰ Factor His the main contributor to HIV evasion of complement mediated lysis.²¹ Murine and human Factor His composed of twenty repetitive units and each unit is approximately sixty amino acids long.²² Neither murine Factor H nor human Factor H is characterized by an alpha helix or a beta pleated sheet. Both human and murine Factor H exists in two different confomiational states (φ₁ and φ₂) that can be separated by hydrophobic chromatography. Both have equal function.²³ Although murine Factor H possesses a high degree of homology to human Factor H, it does not bind to the HIV virus. Establishing an effective HIV infection in a murine model would require the assimilation of human Factor H.

A variety of sialic acids (characterized by a 9 carbon backbone) and/or a glycan chain (composed of mostly 5 and 6 carbon sugars) are expressed on the surfaces of animals, fungi, plants, protozoa, bacteria and viruses. Mammals possess a variety of sialic acid recognizing proteins known as Siglecs. To date, eleven functional Siglecs and one Siglecs like molecule (Siglec L1) have been characterized. Macrophages express Siglec 1 (sialoadhesin), B cells express Siglec 2 (CD22) and monocytes express Siglec 3 (CD33). Cells involved in the innate immune response including natural killer cells and granulocytes are characterized by Siglecs 1, 3, 5, 7 and 10. The function of a protein and its potential immunogenicity are in part related to its surface glycan or sialic acid residues. Therefore, a potential rejection and function issue exists if proteins from animals expressing different surface sugar molecules co-exist in the same animal. Interestingly, the CMP-Neu5Ac synthetase genes that encode the enzymatic machinery necessary for sialic acids are found with one exception only in fruit flies, rainbow trout, mice and humans. Surprisingly, one bacteria Streptomyces coelicolor also expresses this genetic machinery. Lateral gene transfer between this bacterium and a eukaryotic host best explains this anomaly.²⁴ Therefore, a murine model obviates this overwhelming concern.

The mucosa of the murine GI tract has been well described. The surface of Peyer's patches is covered by epithelium associated with a variety of lymphoid cells known as the follicle-associated epithelium (FAE). The FAE is composed of a variety of cells including cells known as M cells. These cells exhibit slender cytoplasmic extensions around lymphoid cells. The basolateral surface of the M cell is deeply invaginated forming a pocket that shortens the distance from the apical to the basolateral surface. The pocket is rich in B cells, T cells, macrophages and dendritic cells. Antigen uptake by M cells does not result in intracellular degradation but rather delivery of the intact molecule to the underlying lymphoid tissue. The apical surface of the M cell lacks the brush border of typical gut lining enterocytes. Furthermore, the M cells are not coated with the thick glycocalyx found on enterocytes. Finally, the distribution of actin-associated protein villin in M cells differs from enterocytes. These characteristics make M cells ideal targets for absorption of proteins produced by recombinant commensal organisms needed for HIV replication.²⁵

A variety of methods will target the M cells for absorption of defined proteins. These include, but are not limited to: (1) cholera toxin-B subunit, (2) carbohydrate lectins, (3) genetically engineered IgA or the secretory component of IgA. Splicing the genetic DNA sequence for a defined protein needed for HIV replication and linking that protein to 1, 2 or 3, above, will target the protein to the M cells and ultimately to the underlying immune tissue.²⁶

Alternatively, attenuated viruses particularly the mouse reovirus, attenuated Poliovirus type 1 and the attenuated Sabin strain selectively adhere to M cells. These viruses can be exploited for transporting a defined protein into Peyer's patches.²⁷

Certain attenuated bacteria also target the M cell apical membrane. These include Vibrio Cholerae, Salmonella, Shigella, Yersinia and BCG. Attenuation of these organisms renders them non-virulent. They can be exploited in targeting recombinant proteins to the M cells and the underlying immune tissue.²⁸

As a final step, the described proteins are administered to the animal by way of its GALT followed by infection of live HIV. Infection with live HIV will result in Tat protein transcription and translation with the resulting Tat mediated immune suppression. Alternatively, Tat protein or the incorporation of the DNA encoding the Tat protein can be administered directly in combination with other proteins or incorporated into the commensal through recombinant technology described above.

### Administration and Supplements

It is possible for the proteins, composition of proteins and or compositions of incorporated DNA encoding the proteins to be administered as a pharmaceutical formulation or preparation, optionally with supplements or other compositions as described above. If protein carriers are used they must be “pharmaceutically acceptable” in the sense of being compatible with the other ingredients of the formulation and not deleterious to the recipient thereof. The coupling of protein carriers (e.g., complement proteins) is known within pharmacology.

Administration may be made in a variety of routes, for example orally, transbucally, transmucosally, sublingually, nasally, rectally, vaginally, intraocularly, intramuscularly, intralymphatically, intravenously, subcutaneously, transdermally, intradermally, intra tumor, topically, transpulmonarily, by inhalation, by injection, or by implantation, etc. Various forms of the composition may include, without limitation, capsule, gel cap, tablet, enteric capsule, encapsulated particle, powder, suppository, injection, ointment, cream, implant, patch, liquid, inhalant, or spray, systemic, topical, or other oral media, solutions; suspensions, infusion, etc. Because some of the first targets for infection with HIV are epithelial cells and Langerhans cells in the skin and rectal mucosa, then a preferable embodiment of delivery is dermal combined with rectal suppositories.

Those skilled in the art will recognize that for administration by injection, formulation in aqueous solutions, such as Ringer's solution or a saline buffer may be appropriate. Liposomes, emulsions, and solvents are other examples of delivery vehicles. Oral administration would require carriers suitable for capsules, tablets, liquids, pills, etc, such as sucrose, cellulose, etc.

The preferred method of administration would be via commensal organisms genetically modified to express one or more human derived proteins needed for HIV replication. The preferred area of administration would be the intestines targeting Peyer's patches. The delivery and deliberate infection of live HIV is well known in the art and includes intra vaginal, rectal and systemic portals.

In conclusion, the present invention provides compositions and a method for producing an animal model for HIV induced disease. The present invention is an animal adapted to simulate a human-like immune response to HIV, which is accomplished by activation and inactivation of complement of proteins within the animal. Accordingly, the present invention stages certain human proteins within an animal by way of its GALT followed by infection of live HIV.

The analysis and development of the animal model for HIV induced disease should incorporate a wide range of doses of the proteins necessary for viral replication and immune evasion, deregulation and/or suppression for evaluation. Animal trials should consider differences in size, species, and immunological characteristics.

The above examples should be considered to be exemplary embodiments, and are in no way limiting of the present invention. Thus, while the description above refers to particular embodiments, it will be understood that many modifications may be made without departing from the spirit thereof.

Prokaryotic organisms lack the post translational modification machinery found in eukaryotic organisms. Yeast such as Saccharomyces cerevisiae are eukaryotes often found as commensal organisms in GALT. Yeast may therefore be preferable as recombinatorial vectors.

A blend of genetic manipulations may yield the optimal animal model. A mouse with one or the other above-mentioned amino acid substitutions in the Cyclin T protein that renders it Tat and TAR processive would be a good starting point. This murine model could then assimilate the CD4 receptor and the CCR5 and CXCR4 co-receptors by transgenic technology. Other proteins the mouse is lacking to affect HIV replication, immune evasion and immune disregulation could be supplied in trans via recombinatorial GALT vectors.

### REFERENCES

1. Lindblad-Toh, et al., 2001, http://www.ncbi.nlm.nih.gov
2. Nagy, Andras, et al., “Manipulating the Mouse Embryo,” A Laboratory Manual, 2003, 3d ed., Ch. 1, pp. 1-29
3. Bieniasz, Paul D., et. al., “Analysis of the Effect of Natural Sequence Variation in Tat and in Cyclin T on the Formation and RNA Binding Properties of Tat-Cyclin T Complexes,” J of Virology, July 1999, Vol. 73, pp. 5777-5786
4. Czerkinsky, Cecil, et. al., “Mucosal immunity and tolerance: relevance to vaccine development,” Immunologic Reviews, 1999, Vol. 170, pp. 197-222
5. Cerkinsky, Cecil, et. al., “Mucosal immunity and tolerance: relevance to vaccine development,” Immunologic Reviews, 1999, Vol. 170, pp. 197-222
6. Czerkinsky, Cecil, et. al, “Mucosal immunity and tolerance: relevance to vaccine development,” Immunologic Reviews, 1999, Vol. 170, pp. 197-222
7. Czerkinsky, Cecil, et. al., “Mucosal immunity and tolerance: relevance to vaccine development,” Immunologic Reviews, 1999, Vol. 170, pp. 197-222
8. Huang, Fang-Ping, et. al., “A Discrete Subpopulation of Dendritic Cells Transports Apoptotic Intestinal Epithelial Cells to T Cell Areas of Mesenteric Lymph Nodes,” J. Exp. Med., 2000, Vol. 191, No. 3, February 7, pp. 435-443
9. Nicholl, Desmond S. T., An Introduction to Genetic Engineering, 2002, 2d ed., Ch. 4, pp. 43-53
10. H Nicholl, Desmond S. T., An Introduction to Genetic Engineering, 2002, 2d ed., Ch. 5, pp. 57-85
11. Nicholl, Desmond S. T., An Introduction to Genetic Engineering, 2002, 2d ed., Ch. 5, pp. 57-85
12. Nicholl, Desmond S. T., An Introduction to Genetic Engineering, 2002, 2d ed., Ch. 5, pp. 57-85
13. Nicholl, Desmond S. T., An Introduction to Genetic Engineering, 2002, 2d ed., Ch. 5, pp. 57-85
14. Nicholl, Desmond S. T., An Introduction to Genetic Engineering, 2002, 2d ed., Ch. 5, pp. 57-85
15. Langel, Ulo, Handbook of Cell-Penetrating Peptides, 2007, 2d ed., Ch. 1, pp. 1-28.
16. Sayah, David. M., et. al., “Cyclophilin A retrotransposition into TRIM5 explain owl monkey resistance to HIV-1,” Nature, 2004, Vol. 430, July 29, pp 569-573
17. Mariani, Roberto, et al., “A Block to Human Immunodeficiency Virus Type 1 Assembly in Murine Cells,” Journal of Virology, Vol. 74, No. 8, April 2000, pp. 3859-3870
18. Bieniasz, Paul D., et al., “Recruitment of a protein complex containing Tat and cyclin T1 to TAR governs the species specificity of HIV-1 Tat,” The EMBO Journal, Vol. 17, 1998, pp. 7056-7065
19. Spear, G. T., et al., “Human immunodeficiency virus (HIV)-infected cells and free virus directly activate the classical complement pathway in rabbit, mouse and guinea-pig sera; activation results in virus neutralization by virolysis,” Immunology, Vol. 73, 1991, pp. 377-382
20. Stoiber, Heribert, et al., “Interaction of several complement proteins with gp120 and gp41, the two envelope glycoproteins of HIV-1,” AIDS, Vol. 9, 1995, pp. 19-26
21. Stoiber, Heribert, et al., “Efficient Destruction of Human Immunodeficiency Virus in Human Serum by Inhibiting the Protective Action of Complement Factor H and Decay Accelerating Factor (DAF, CD55),” J. Exp. Med, January 1996, Vol. 183, pp. 307-310
22. Kristensen, Torsten, et al., “Murine protein His comprised of 20 repeating units, 61 amino acids in length,” Proc. Nat. Acad. Sci., USA, Vol. 83, June 1986, pp. 3963-3967
23. Ripoche, Jean, et al., “The complete amino acid sequence of human complement factor H,” Biochem. J., Vol. 249, 1988, pp. 593-602
24. Angata, Takashi, et al., “Chemical Diversity in the Sialic Acids and Related α-Keto Acids: An Evolutionary Perspective,” Chem. Rev., Vol. 102, 2002, pp. 439-469
25. Kiyono, Horishi, et al., Mucosal Vaccines, 1996, Ch. 2, pp. 17-40 and Ch. 3, pp. 41-53
26. Kiyono, Horishi, et al., Mucosal Vaccines, 1996, Ch. 3, pp. 41-53
27. Kiyono, Horishi, et al., Mucosal Vaccines, 1996, Ch. 3, pp. 41-53
28. Kiyono, Horishi, et al., Mucosal Vaccines, 1996, Ch. 3, pp. 41-53
29. S., Bounou, et al., The importance of virus-associated host ICAM-1 in human immunodeficiency virus type 1 dissemination depends on the cellular context, FASEB J. 2004 August; 18(11):1294-6. Epub 2004 Jun. 18

### APPENDIX A

The following information is generally known by those in the art and can be found in medical texts generally including by way of example, Mucosal Vaccines, Hematology Basic Principles and Practices, and Immunology, Infection and Immunity and journals such as Immunologic Reviews, Nature, Virology, and Molecular Immunology.

HIV-1

| | FUNCTION(S) OF | | |
|---|---|---|---|
| | HOST | COMPONENT | FUNCTION(S) OF |
| HOST | MOLECULE IN HOST | OF INTACT | HOST MOLECULE IN |
| MOLECULE | CELL | VIRION | HIV VIRION |
| Membrane | A type 1 transmembrane | Yes, envelope | Protects virion against |
| Cofactor of | protein present on | | complement cascade. |
| Proteolysis | thymocytes, T cells, B | | Mechanism employed |
| (MCP/CD46) | cells, natural killer cells, | | by HIV identical to that |
| | monocytes, neutrophils, | | found on host cellular |
| | platelets, endothelial | | proteins. |
| | cells, epithelial cells, | | |
| | fibroblasts and placenta. | | |
| | Complement control | | |
| | protein prevents | | |
| | convertase (C4b, 2b or | | |
| | C3 convertase and C4b, | | |
| | 2b, 3b or C5 convertase) | | |
| | production by cleaving | | |
| | C3b into iC3b, an | | |
| | inactive protein. | | |
| | Therefore controls both | | |
| | classical and alternative | | |
| | complement pathways. | | |
| | Cofactor for factor I. | | |
| | Found on chromosome 1 | | |
| | location 1q32. Has a | | |
| | role in tailoring innate | | |
| | immune recognition of | | |
| | apoptotic and necrotic | | |
| | cells. Bridges innate | | |
| | and acquired immunity | | |
| | by regulating T cell- | | |
| | induced inflammatory | | |
| | reactions. | | |
| Decay | A | Yes, envelope | Protects virion against |
| Accelerating | glycosylphosphatidylinositol | | complement cascade. |
| Factor | (GPI)-anchored | | Mechanism employed |
| (DAF/CD55) | membrane protein | | by HIV identical to that |
| | present on all | | found on host cellular |
| | hemopoietic cells. | | proteins. |
| | Complement control | | |
| | protein competes with | | |
| | factor B for binding to | | |
| | C3b on the cell surface | | |
| | and displaces Bb protein | | |
| | from a convertase C3 or | | |
| | C5 convertase that has | | |
| | already formed. Found | | |
| | on chromosome 1 | | |
| | location 1q32. Antigen- | | |
| | presenting cell | | |
| | exosomes are protected | | |
| | from complement- | | |
| | mediated lysis by | | |
| | expression of CD55 and | | |
| | CD59. | | |
| 20-kd | A GPI-anchored | Yes, envelope | Protects virion against |
| homologous | membrane present on | | complement cascade. |
| restriction factor | many hemopoietic cells. | | Mechanism employed |
| (HRF-20/CD59) | Complement control | | by HIV identical to that |
| | protein inhibits cell | | found on host cellular |
| | surface membrane | | proteins. |
| | attack complex. | | |
| | Recognizes specific | | |
| | domains within C8 and | | |
| | C9. Found on | | |
| | chromosome 11 location | | |
| | 11p13. Antigen- | | |
| | presenting cell | | |
| | exosomes are protected | | |
| | from complement- | | |
| | mediated lysis by | | |
| | expression of CD55 and | | |
| | CD59. | | |
| Factor H | Complement control | Yes, envelope | Protects virion against |
| | protein inhibits C3 and | | complement cascade. |
| | C5 convertase formation | | Mechanism employed |
| | and promotes | | by HIV identical to that |
| | degradation of C3 | | found on host cellular |
| | convertase. Found on | | proteins. |
| | chromosome 1 location | | |
| | 1q32. Factor H domains | | |
| | 19-20 alone are capable | | |
| | of discriminating | | |
| | between host-like and | | |
| | complement-activating | | |
| | cells. Three heparin- | | |
| | binding sites were | | |
| | identified in complement | | |
| | factor H1. Factor H is | | |
| | cleaved by a dermatan | | |
| | sulfate-mediated | | |
| | protease identified in | | |
| | blood. The site and | | |
| | putative residues on | | |
| | factor H (FH) essential | | |
| | for the interaction of the | | |
| | C-terminal end of FH | | |
| | with C3d, C3b, and | | |
| | heparin have been | | |
| | identified; the heparin- | | |
| | and C3d-binding sites | | |
| | are overlapping. | | |
| Thy-1 (CD90) | A GPI-anchored | Yes, envelope | HIV-1 Matrix co- |
| | membrane protein | and Matrix | localizes with Thy-1 in |
| | present on | protein | lipid rafts, the site of |
| | prothymocytes, brain | | virus particle budding |
| | and other non lymphoid | | from cells, and Thy-1 is |
| | tissue. Found on | | incorporated into virus |
| | chromosome 11 location | | particles as a result of |
| | 11q22.3-q23. Role of | | this process. |
| | endothelial cell receptor | | |
| | Thy1 in cell adhesion | | |
| | has been defined. | | |
| | AlphaX-beta2 | | |
| | specifically interacts with | | |
| | Thy-1. Thy-1 and Mac-1 | | |
| | interact and are involved | | |
| | in the adhesion of | | |
| | leukocytes to activated | | |
| | endothelial cells as well | | |
| | as in subsequent trans | | |
| | endothelial migration of | | |
| | leukocytes into the | | |
| | perivascular tissue. | | |
| GM1 (β- | Gangliosides are | Yes, envelope | May facilitate |
| galactosidase) | glycosphingolipids found | | gp120/gp41/CD4 |
| | in neuronal and synaptic | | membrane fusion. |
| | membranes. Basic | | |
| | structure consists of an | | |
| | oligosaccharide chain | | |
| | attached to a hydroxyl | | |
| | (OH⁻) group of ceramide | | |
| | and sialic acid bound to | | |
| | galactose. Gangliosides | | |
| | are degraded | | |
| | sequentially by specific | | |
| | exoglycosidases. Found | | |
| | on chromosome 3 | | |
| | location 3p21.33. | | |
| | Catalyzes the enzymatic | | |
| | conversion of gal- | | |
| | NAcglc-gal-glc- | | |
| | ceramide→NAcglc-gal- | | |
| | glc-ceramide. Involved | | |
| | in cell-cell interaction, | | |
| | signal transduction, and | | |
| | cell activation. | | |
| HLA-DR | Antigen presentation, | Yes, envelope | Interacts with CD4 |
| | MHC class II directly | | glycoprotein on target |
| | presents peptide | | cells. Without |
| | antigens to CD4 T cells. | | associated antigen in |
| | Highly polymorphic. | | the peptide binding |
| | Heterodimer consisting | | cleft of HLA-DR and |
| | of an alpha (DRA) and a | | co-stimulating |
| | beta (DRB) chain, both | | molecular interactions, |
| | anchored in the | | CD4 cell will be |
| | membrane. Presents | | rendered anergic. |
| | peptides derived from | | HIV-1 Gag expression |
| | extracellular proteins by | | is able to induce HLA- |
| | antigen presenting cells, | | DR cell-surface |
| | B cells, dendritic cells | | localization in H78- |
| | and macrophages. | | C10.0 cells. In human |
| | Found on chromosome 6 | | macrophages, HIV-1 |
| | location 6p21.3. | | Gag proteins co- |
| | | | localize with MHC II |
| | | | (HLA-DR), CD63, and |
| | | | Lamp1 in MHC II |
| | | | compartments. HIV-1 |
| | | | Capsid (p24) inhibits |
| | | | interferon gamma |
| | | | induced increases in |
| | | | HLA-DR and |
| | | | cytochrome B heavy |
| | | | chain mRNA levels in |
| | | | the human monocyte- |
| | | | like cell line THP1. |
| | | | HIV-1 Tat down |
| | | | regulates expression of |
| | | | MHC class II genes in |
| | | | antigen-presenting |
| | | | cells (APC) by |
| | | | inhibiting the |
| | | | transactivator of MHC |
| | | | class II genes, CIITA. |
| | | | HIV-1 Tat up regulates |
| | | | HLA-DR expression in |
| | | | monocyte-derived |
| | | | dendritic cells and T |
| | | | cells, thereby driving T |
| | | | cell-mediated immune |
| | | | responses and |
| | | | activation. Associates |
| | | | with HIV-1 gp41. |
| | | | Enhances HIV-1 |
| | | | infectivity. Not affected |
| | | | by viral tropism which |
| | | | is determined by the |
| | | | V3 loop of gp120. |
| | | | Amino acids 708-750 |
| | | | of gp41 required for |
| | | | MHC-II incorporation |
| | | | into the HIV-1 |
| | | | envelope. |
| | | | Approximately 375 to |
| | | | 600 molecules of HLA |
| | | | II are incorporated into |
| | | | each HIV-1 virion. |
| | | | HLA II DR is the |
| | | | predominant if not only |
| | | | subtype of HLA II |
| | | | detected on the |
| | | | surface of most HIV-1 |
| | | | virions. Therefore, |
| | | | HLA II DR is |
| | | | selectively |
| | | | incorporated into the |
| | | | viral envelope. |
| ICAM-1 | A type 1 transmembrane | Yes, envelope | Increases HIV |
| (Intercellular | protein present on | | infectivity by a factor of |
| adhesion | leukocytes and | | 5 to 10 in T cells not |
| molecule 1 also | endothelial cells and | | expressing the LFA |
| known as CD54) | inducible on | | (lymphocyte function- |
| ICAM-2 ICAM-3 | lymphocytes, dendritic | | associated antigen)-1 |
| | cells, keratinocytes, | | ligand. In T cells |
| | chondrocytes, | | expressing the LFA-1 |
| | fibroblasts, and epithelial | | ligand, infectivity |
| | cells. A ligand for CD11 | | increases one hundred |
| | and CD18. Adhesion | | fold. ICAM-1 |
| | molecule binds to LFA-1 | | increases the stability |
| | (lymphocyte function | | of virion/cell |
| | antigen 1 also known as | | interaction. The ICAM- |
| | CD11a/CD18) | | 1/LFA-1 association |
| | contributes to T and B | | was a more efficient |
| | cell activation. Found on | | transmission factor for |
| | chromosome 19 location | | HIV-1 bearing ICAM-1 |
| | 19p13.3-p13.2. | | than combined |
| | Signaling via ICAM-1 | | gp120/DC-SIGN & |
| | induces adhesiveness of | | ICAM-3/DC-SIGN. The |
| | mononuclear | | finding was confirmed |
| | phagocytes. Direct | | in human lymphoid |
| | interaction of ICAM-1 | | tissues.²⁹ ICAM-1 co- |
| | with cytoplasmic alpha- | | localizes with HIV-1 |
| | actinin-1 and -4 is | | Matrix at sites of cell- |
| | essential for leukocyte | | to-cell membrane |
| | extravasation. Functions | | contact and is |
| | with VCAM-1 (vascular | | incorporated into virus |
| | cell adhesion molecule- | | particles. Expression |
| | 1), activated moesin, | | of HIV-1 Nef in |
| | actin, alpha-actinin, | | macrophages induces |
| | vinculin, ezrin and VASP | | the release of soluble |
| | (vasodilator-stimulated | | ICAM, which |
| | phosphoprotein) to | | upregulates the |
| | facilitate leukocyte | | expression of co- |
| | adhesion. An inducible | | stimulatory receptors |
| | ligand for both LFA-1 | | on B lymphocytes. |
| | (αLβ2) and Mac-1 | | Interacts with Tat and |
| | (αMβ2). Interacts with | | Nef. Tat up regulates |
| | the actin cytoskeleton | | the expression of |
| | using ezrin as an | | ICAM-1 on endothelial |
| | intermediate. | | cells and astrocytes, in |
| | | | part regulated by |
| | | | NF_{K}B. May be |
| | | | correlated with the |
| | | | development of AIDS- |
| | | | related Kaposi's |
| | | | sarcoma. Increases |
| | | | HIV infectivity. |
| | | | Interacts directly or |
| | | | indirectly with Gag |
| | | | polyprotein precursor |
| | | | Pr55^{Gag} in newly |
| | | | formed virions. May |
| | | | allow virion fusion with |
| | | | CD4 negative cells. |
| LFA-1 | A type I transmembrane | Yes, envelope | The incorporation of |
| (CD11A/LFA1A/I | protein found on | | adhesion molecules |
| TGAL [integrin, | lymphocytes, | | may allow virion fusion |
| alpha L (antigen | neutrophils, monocytes, | | with CD4 negative |
| CD11A [p180]), | and macrophages, | | cells. |
| lymphocyte | facilitates cell adhesion | | |
| function- | and cell activation. | | |
| associated | Contributes to B cell/T | | |
| antigen 1; alpha | cell interactions. Natural | | |
| polypeptide] | ligand, ICAM-1. Found | | |
| | on chromosome 16 | | |
| | location 16p11.2. NK | | |
| | cells receive early | | |
| | activation signals directly | | |
| | through LFA-1 without | | |
| | co-stimulatory signals. | | |
| VCAM-1 | A member of the Ig | Yes, envelope | B-lymphomas |
| (vascular cell | superfamily, a cell | | characterize HIV. |
| adhesion | surface sialoglycoprotein | | Endothelial cells (EC's) |
| molecule 1; also | expressed by cytokine- | | bind firmly to malignant |
| known as | activated endothelial | | B cells. The key event |
| CD106) | cells, macrophages, | | promoting EC-BL-cell |
| | dendritic cells and | | adhesion was Vpu of |
| | marrow stroma. Type I | | HIV-1 upregulates |
| | membrane protein | | endothelial CD40, |
| | mediating leukocyte- | | facilitating increased |
| | endothelial cell adhesion | | expression of vascular |
| | and signal transduction. | | cell adhesion molecule |
| | Two alternatively spliced | | 1 (VCAM-1). |
| | transcripts encoding | | Therefore, Vpu may |
| | different isoforms have | | enhance the metastatic |
| | been described. | | potential of B |
| | Interacts with α₄β₁ | | lymphomas. |
| | (VCAM-1 counterpart or | | |
| | ligand). Found on | | |
| | chromosome 1 location | | |
| | 1p32-p31. | | |
| VLA-4 | | Yes, envelope | |
| (CD29/CD49d) | | | |
| MHC-1 | In humans, six MHC | Yes, envelope | Enhances HIV |
| | class 1 isotypes have | | infectivity and changes |
| | been identified: HLA-A, | | gp120 conformation. |
| | HLA-B, HLA-C, HLA-E, | | Without antigen in |
| | HLA-F and HLA-G. | | MHC-1 binding groove |
| | HLA-A, HLA-B and HLA- | | and co-stimulatory |
| | C function to present | | activity, anergy results. |
| | antigens to CD8 T cells | | HIV-1 Nef down |
| | and to form ligands for | | regulates surface |
| | natural killer (NK) cell | | expression of CD4 and |
| | receptors. HLA-E and | | MHC-1 in resting CD4⁺ |
| | HLA-G also ligands for | | T lymphocytes. Nef up |
| | NK-cell receptors. HLA- | | regulates cell surface |
| | A is found on | | levels of the MHC-2 |
| | chromosome 6 location | | invariant chain CD74. |
| | 6p21.3. | | Nef down regulates |
| | | | HLA class I expression |
| | | | and therefore |
| | | | suppresses the |
| | | | cytolytic activity of HIV- |
| | | | 1-specific cytotoxic T- |
| | | | lymphocyte (CTL) |
| | | | clones. Macrophage- |
| | | | tropic (M-tropic) HIV-1 |
| | | | Nef down regulates |
| | | | expression of HLA-A2 |
| | | | on the surface of |
| | | | productively infected |
| | | | macrophages. HIV-1 |
| | | | group N and group O |
| | | | Nef weakly down |
| | | | regulates CD4, CD28, |
| | | | and class I and II MHC |
| | | | molecules and up |
| | | | regulates surface |
| | | | expression of the |
| | | | invariant chain (Ii) |
| | | | associated with |
| | | | immature major |
| | | | histocompatibility |
| | | | complex (MHC) class |
| | | | II. Nef interrupts MHC- |
| | | | I trafficking to the |
| | | | plasma membrane and |
| | | | inhibits antigen |
| | | | presentation. Nef |
| | | | interacts with the μ1 |
| | | | subunit of adaptor |
| | | | protein (AP) AP-1A, a |
| | | | cellular protein |
| | | | complex implicated in |
| | | | TGN linking |
| | | | endosome/lysosome |
| | | | pathways. HIV-1 Nef |
| | | | binds to the MHC-I |
| | | | (HLA-A2) hypo |
| | | | phosphorylated |
| | | | cytoplasmic tails in the |
| | | | endoplasmic reticulum; |
| | | | this Nef-MHC-I |
| | | | complex migrates into |
| | | | the Golgi apparatus |
| | | | then into the lysosomal |
| | | | compartments for |
| | | | degradation. Nef |
| | | | promotes a physical |
| | | | interaction between |
| | | | endogenous AP-1 and |
| | | | MHC-I. The Pro-X-X- |
| | | | Pro motif in HIV-1 Nef |
| | | | induces the |
| | | | accumulation of CCR5 |
| | | | (HIV-1 M-tropic |
| | | | coreceptor) in a |
| | | | perinuclear |
| | | | compartment where |
| | | | both molecules co- |
| | | | localize with MHC-1. |
| | | | The Pro-X-X-Pro motif |
| | | | interacts with src |
| | | | homology region-3 |
| | | | domains of src-like |
| | | | kinases interfering with |
| | | | cell signaling |
| | | | pathways. HIV-1 Nef |
| | | | selectively down |
| | | | regulates HLA-A and |
| | | | HLA-B but does not |
| | | | significantly affect |
| | | | HLA-C or HLA-E, |
| | | | which allows HIV- |
| | | | infected cells to avoid |
| | | | NK cell-mediated lysis. |
| | | | Nef decreases the |
| | | | incorporation of MHC-1 |
| | | | molecules into virions. |
| | | | Furthermore, Nef down |
| | | | regulates MHC-1 |
| | | | expression on human |
| | | | dendritic cells. |
| | | | Therefore, HIV-1 Nef |
| | | | impairs antigen |
| | | | presentation to HIV- |
| | | | specific CD8+ T |
| | | | lymphocytes. HIV-1 |
| | | | Nef-induced down |
| | | | regulation of MHC-I |
| | | | expression and MHC-I |
| | | | targeting to the trans- |
| | | | Golgi network (TGN) |
| | | | require the binding of |
| | | | Nef to PACS-1 |
| | | | (phosphofurin acidic |
| | | | cluster sorting protein |
| | | | 1). PACS-1 is a |
| | | | protein with a putative |
| | | | role in the localization |
| | | | of proteins to the trans- |
| | | | Golgi network (TGN) |
| | | | including furin which |
| | | | cleaves gp160. HIV-1 |
| | | | Nef down regulates |
| | | | MHC-1 on lymphoid, |
| | | | monocytic and |
| | | | epithelial cells. Nef |
| | | | expression results in |
| | | | rapid internalization |
| | | | and accumulation of |
| | | | MHC-1 in endosomal |
| | | | vesicles which degrade |
| | | | MHC-1 molecules. Nef |
| | | | blocks transport of |
| | | | MHC-I molecules to |
| | | | the cell surface, |
| | | | leading to |
| | | | accumulation of MHC- |
| | | | 1 in intracellular |
| | | | organelles. |
| | | | Furthermore, the effect |
| | | | of Nef on MHC-1 |
| | | | molecules (but not on |
| | | | CD4) requires |
| | | | phosphoinositide 3- |
| | | | kinase (PI 3-kinase) |
| | | | activity found on the |
| | | | cytoplasmic side of the |
| | | | plasma membrane. |
| CD63 | A type III | Yes, envelope | The efferent arm of |
| | transmembrane protein | | viral replication occurs |
| | present on activated | | in the endosomes. |
| | platelets, monocytes, | | The CD63 marker is |
| | macrophages, and in | | the result of the |
| | secretory granules of | | endosomal sorting |
| | vascular endothelial | | machinery and |
| | cells. Facilitates | | facilitates further |
| | adhesion to activated | | endosomal viral |
| | endothelium. A marker | | maturation. CD63 may |
| | of late endosomes. | | facilitate HIV-1 |
| | Found on chromosome | | penetration of |
| | 12 location 12q12-q13. | | macrophages. |
| | Regulates cell | | |
| | development, activation, | | |
| | growth and motility. | | |
| | CD63 represents an | | |
| | activation-induced | | |
| | reinforcing element, | | |
| | whose triggering | | |
| | promotes sustained and | | |
| | efficient T cell activation | | |
| | and expansion. CD63 | | |
| | serves as an adaptor | | |
| | protein that links its | | |
| | interaction partners to | | |
| | the endocytic machinery | | |
| | of the cell. | | |
| CD81 | A type III | Yes, envelope | The efferent arm of |
| | transmembrane protein | | viral replication occurs |
| | found on lymphocytes | | in the endosomes. |
| | which facilitates signal | | The CD81 marker is |
| | transduction. A marker | | the result of the |
| | of late endosomes. | | endosomal sorting |
| | Found on chromosome | | machinery and |
| | 11 location 11p15.5. | | facilitates endosomal |
| | CD81 signaling events | | viral maturation. |
| | could be mediated by | | |
| | 14-3-3 adapter proteins, | | |
| | and these signals may | | |
| | be dependent on cellular | | |
| | redox. 14-3-3 Proteins | | |
| | recognize | | |
| | phosphoserine/threonine | | |
| | amino acids in specific | | |
| | primary amino acid | | |
| | sequences. Control cell | | |
| | cycle, apoptosis, gene | | |
| | transcription, DNA | | |
| | replication and | | |
| | chromatin remodeling. | | |
| CD82 | A type III | Yes, envelope | The efferent arm of |
| | transmembrane protein | | viral replication occurs |
| | present on epithelial | | in the endosomes. |
| | cells, endothelial cells, | | The CD82 marker is |
| | and activated | | the result of the |
| | lymphocytes. May be | | endosomal sorting |
| | involved in intracellular | | machinery and |
| | calcium fluctuations. A | | facilitates endosomal |
| | marker of late | | viral maturation. HIV-1 |
| | endosomes. Found on | | Gag proteins co- |
| | chromosome 11 location | | localize with the type III |
| | 11p11.2. CD82 | | transmembrane |
| | facilitates transcription of | | proteins CD9, CD81, |
| | IL-2 gene. Coordinates | | CD82 and CD63. |
| | activity with β1 integrin | | |
| | in IL-2 gene | | |
| | transcription. | | |
| CD107a (LAMP- | A type I transmembrane | Yes, envelope | The efferent arm of |
| 1 [Lysosome- | protein present on | | viral replication occurs |
| associated | activated platelets. A | | in the endosomes. |
| membrane | marker of late | | The LAMP-1 marker is |
| glycoprotein 1 | endosomes. Found on | | the result of the |
| precursor]) | chromosome X location | | endosomal sorting |
| | Xp21.1. | | machinery and |
| | | | facilitates endosomal |
| | | | viral maturation. |
| HP68 | RNase L inhibitor. A | Present in | Interacts with HIV-1 |
| | member of the | immature | Gag. Protects viral |
| | superfamily of ATP- | capsid | RNA from intracellular |
| | binding cassette (ABC) | assembly | RNAse degradation. |
| | transporters. ABC | intermediates. | Also interacts with Vif. |
| | proteins transport | | Essential for post- |
| | various molecules | | translational events in |
| | across extra- and intracellular | | immature HIV-1 capsid |
| | membranes. | | assembly. Interaction |
| | Inhibits protein synthesis | | of Vif involved in virion |
| | in the 2-5A/RNase L | | morphogenesis and |
| | system, the central | | infectivity. Basic |
| | pathway for viral | | residues in NC recruit |
| | interferon action. Two | | both viral RNA and |
| | transcript variants | | HP68 facilitating |
| | encoding the same | | capsid assembly. |
| | protein have been found | | |
| | for this gene. Found on | | |
| | chromosome 4 location | | |
| | 4q31. | | |
| Ezrin (villin 2) | Cytoskeletal protein | Yes, virion, | Facilitates viral fusion |
| | linking the actin | specifically | with target cell and |
| | cytoskeleton with the | internal | possibly endocytosis of |
| | plasma membrane. | nucleocapsid | virion. Incorporated |
| | Found on chromosome 6 | and reverse | into HIV-1 particles via |
| | location 6q25.2-q26. | transcription | interaction with actin |
| | Binds directly to CD95 | complex | which binds to the p7 |
| | (APO-1/Fas) mediating | | domain of HIV-1 Gag. |
| | apoptosis in CD4⁺ T | | |
| | cells. Part of the | | |
| | ezrin/radixin/moesin | | |
| | (ERM) family proteins. | | |
| | Links the actin | | |
| | cytoskeleton to the | | |
| | dystroglycan adhesion | | |
| | receptor complex. | | |
| | Functions in cell | | |
| | adhesion, cell survival | | |
| | and motility. Function | | |
| | regulated by | | |
| | phosphorylation on two | | |
| | tyrosine residues, one at | | |
| | the amino-terminal, the | | |
| | other in the carboxyl- | | |
| | terminal domain. | | |
| | Involved in signal | | |
| | transduction pathways | | |
| | that involve tyrosine | | |
| | kinases, including PI3K | | |
| | (phosphatidyl inositide 3- | | |
| | kinase) and c-Src (the | | |
| | proto-oncogene of Src | | |
| | tyrosine kinase). | | |
| Moesin | Cytoskeletal protein | Yes, virion, | Facilitates viral fusion |
| | component of the ERM | specifically | and PIC directional |
| | protein family. Localizes | internal | translocation into the |
| | beneath the cell | nucleocapsid | nucleus. Incorporated |
| | membrane and cross | and reverse | into HIV-1 particles via |
| | links the plasma | transcription | interaction with actin |
| | membrane and the | complex | which binds to the p7 |
| | cortical actin | | domain of HIV-1 Gag. |
| | cytoskeleton. Involved | | |
| | in cell adhesion and | | |
| | motility. Widely | | |
| | expressed in B and T | | |
| | cells. As with Ezrin, | | |
| | phosphorylation of both | | |
| | the N- and C-terminal | | |
| | domains serves as | | |
| | activating signals. | | |
| | Moesin interacts with | | |
| | CD43, CD44, CD50 and | | |
| | other proteins containing | | |
| | the PDZ (PSD-95, DIgA | | |
| | and ZO-1) dimerization | | |
| | domain. Found on | | |
| | chromosome X location | | |
| | Xq11.2-q12. | | |
| Actin (beta and | Cytoskeletal protein, | Yes, virion, | An intact actin |
| gamma) | most abundant protein in | specifically | cytoskeleton of host |
| | mammalian cells, up to | internal | cell is essential for |
| | 15% of the total protein | nucleocapsid | efficient reverse |
| | content, highly | and reverse | transcription of HIV-1. |
| | conserved among | transcription | The viral proteins Rev |
| | species. Three major | complex | and Vpr effect actin |
| | isoforms have been | | polymerization |
| | identified - alpha, beta | | facilitating the |
| | and gamma. Alpha | | preintegration complex |
| | predominantly in muscle | | (PIC) entry into the |
| | tissue. Beta and gamma | | nucleus. The matrix |
| | are ubiquitous. Multiple | | protein, p17, also |
| | functions including | | interacts with actin in |
| | changes in cell structure, | | the PIC. Actin is |
| | pliability and motility. | | necessary for the |
| | Actin depolymerizing | | clustering of the HIV |
| | factor (ADF)/cofilin and | | CD4 receptor and the |
| | gelsolin in actin-filament | | CXCR4 co-receptor |
| | are primarily responsible | | with gp120 binding. |
| | for remodeling the actin | | Interaction between |
| | cytoskeleton. | | Nef, Actin and Vav, a |
| | ADF/cofilins are also | | guanine nucleotide |
| | necessary for | | exchange factor of |
| | cytokinesis. Involved in | | Cdc42 and Rac (two |
| | cellular mitosis. Intra | | small GTPases |
| | cellular cytoplasmic | | regulating the actin |
| | streaming is largely | | cytoskeleton) modify |
| | dependent upon actin. | | the actin cortex before |
| | Endocytosis, | | viral budding. N- |
| | phagocytosis and | | terminal myristoylated |
| | pinocytosis are actin | | HIV-1 Nef associates |
| | dependent. Beta actin | | with actin in human B |
| | found on chromosome 7 | | and T lymphocytes. |
| | location 7p15-p12. | | This influences the |
| | Gamma actin found on | | subcellular localization |
| | chromosome 17 location | | of Nef. Nuclear beta- |
| | 17q25. | | actin bundles may be |
| | | | involved in the Rev- |
| | | | dependent |
| | | | nuclear/cytoplasmic |
| | | | transport of intron- |
| | | | containing (unspliced |
| | | | and incompletely |
| | | | spliced) HIV-1 gag |
| | | | mRNA. |
| Ubiquitin | Vesicular transport | Yes, virion | Monoubiquitination of |
| | protein, 76 amino acid | | late domains (L |
| | protein ubiquitous in all | | domains) of viral |
| | mammalian cells | | proteins targets the |
| | correlated with multiple | | proteins to the host |
| | cellular functions, | | intracellular endocytic |
| | including, but not limited | | pathway. HIV-1 L |
| | to, degradation of | | domain is a highly |
| | proteins under | | conserved Pro-Thr- |
| | conditions of stress, | | Ala-Pro (PTAP) |
| | degradation of | | sequence in the p6 |
| | denatured or damaged | | domain of Gag. |
| | proteins, targeted | | |
| | degradation of regulatory | | |
| | proteins, transmembrane | | |
| | receptors, mitotic | | |
| | cyclins, transcription | | |
| | activating proteins, | | |
| | modulation of cell | | |
| | surface receptor activity, | | |
| | import of proteins into | | |
| | cellular organelles, DNA | | |
| | repair, processing and | | |
| | presenting of antigens | | |
| | and ribosomal assembly. | | |
| | Monoubiquitination of | | |
| | plasma membrane | | |
| | receptors targets | | |
| | intracellular proteins to | | |
| | the endocytic pathway | | |
| | and functions as a | | |
| | sorting signal directing | | |
| | the movement of | | |
| | proteins between | | |
| | different endocytic | | |
| | compartments. | | |
| Pin1 (protein | A parvulin, a peptidyl- | Yes, virion | Increases intra cellular |
| [peptidyl prolyl | prolyl isomerase binds to | | NF_{K}B levels. NF_{K}B |
| cis/trans | phosphoserine-proline | | binding sites are found |
| isomerase] NIMA | and phosphothreonine- | | in the HIV-1 core |
| [never in mitosis | proline motifs, essential | | enhancer. |
| gene a)-related | in mitosis, facilitates | | |
| kinase]- | proline cis/trans | | |
| interacting 1) | isomerizations and | | |
| | subsequent tertiary and | | |
| | quaternary protein | | |
| | structures. Proline | | |
| | isomerization of cell | | |
| | cycle protein Cdc25 | | |
| | phosphatase facilitates | | |
| | dephosphorylation of | | |
| | phosphorylated Cdc25 | | |
| | protein by the protein | | |
| | phosphatase PP2A. | | |
| | Found on chromosome | | |
| | 19 location 19p13. | | |
| | Mediates GM-CSF | | |
| | production. Binds c-Fos | | |
| | through specific pS/T-P | | |
| | sites within the c-Fos | | |
| | TAD (carboxyl terminal | | |
| | transactivation domain) | | |
| | resulting in enhanced | | |
| | transcriptional response | | |
| | of c-Fos to polypeptide | | |
| | growth factors that | | |
| | stimulate ERK | | |
| | (extracellular regulated | | |
| | kinases). Involved in the | | |
| | cooperative activity of c- | | |
| | Jun and c-Fos to | | |
| | regulate AP-1- | | |
| | dependent gene | | |
| | transcription upon | | |
| | phosphorylation by | | |
| | mitogen-activated kinase | | |
| | (MAPK) family members. | | |
| | Binds to the pThr254- | | |
| | Pro motif in p65 and | | |
| | inhibits p65 binding to | | |
| | I_{K}Bα, resulting in | | |
| | increased nuclear | | |
| | accumulation and | | |
| | protein stability of p65 | | |
| | and enhanced NF_{K}B | | |
| | activity. Interacts with | | |
| | transcription factor β- | | |
| | catenin (cadherin- | | |
| | associated protein) and | | |
| | increases the | | |
| | transcription activity of | | |
| | cyclin D1. Interacts with | | |
| | the carboxyl terminal | | |
| | domain (CTD) of RNA | | |
| | polymerase II (RNAPII). | | |
| | Inhibits the CTD | | |
| | dephosphorylation by | | |
| | FCP1. Enhances the | | |
| | phosphorylation of the | | |
| | CTD domain of RNAP II | | |
| | by the cdc2/cyclinB | | |
| | complex. Co-localizes | | |
| | with the splicing factor | | |
| | SC35 in the cell nucleus. | | |
| | These protein | | |
| | aggregates known as | | |
| | “speckles” contain | | |
| | transcription factors and | | |
| | pre-mRNA. Speckles | | |
| | are intra nuclear | | |
| | warehouses storing | | |
| | components of | | |
| | transcription and the | | |
| | RNA editing. | | |
| tRNA synthetase | Ligase, charges or | Yes, virion | tRNA^{lys3} binds to the |
| or aminoacyl | aminoacylates key RNA | | primer binding site |
| tRNA synthetase | molecules linking the | | initiating reverse |
| | molecule to the | | transcription. In HIV-1 |
| | respective amino acid. | | an RNA loop formed |
| | One synthetase for each | | by the tRNA^{lys3} |
| | amino acid found in | | anticodon and an |
| | mammalian cells. ATP | | adenine rich RNA loop |
| | dependent. | | initiates reverse |
| | | | transcription. |
| tRNA^{lys} | Allows incorporation of | Yes, virion | Induces three |
| | lysine into proteins by | associated | dimensional structural |
| | the host translational | attached to | changes in the |
| | apparatus. | primer binding | unspliced viral RNA to |
| | | site (PBS) | allow reverse |
| | | | transcription to |
| | | | proceed. |
| GAPDH | In glycolysis, | Yes, virion | ?????? |
| (Glyceraldehyde- | enzymatically converts | | |
| 3-phosphate | Glyceraldehyde-3- | | |
| dehydrogenase) | phosphate to 1,3- | | |
| | bisphosphoglycerate. | | |
| | Also involved in cell | | |
| | cycle regulation by | | |
| | modulating cyclin B- | | |
| | cdk1, apoptosis, | | |
| | membrane fusion, | | |
| | microtubule bundling, | | |
| | phosphotransferase | | |
| | activity, nuclear RNA | | |
| | export, programmed | | |
| | neuronal cell death, DNA | | |
| | replication, and DNA | | |
| | repair. Found on | | |
| | chromosome 12 location | | |
| | 12p13. | | |
| MAPK/ERK2 | Serine/threonine kinases | Yes, virion | Phosphorylates |
| (mitogenic | important in regulation of | | p6(gag), involved in |
| activated protein | growth and cellular | | the budding stage of |
| kinase/extracellular | differentiation via a | | HIV-1 life cycle. MAPK |
| regulated | cascade of sequential | | (ERK1 and ERK2) |
| kinases) | protein kinases and | | regulates HIV-1 |
| | scaffold proteins. Found | | infectivity by |
| | on chromosome 22 | | phosphorylating Vif. |
| | locations 22q11.2; | | |
| | 22q11.21. | | |
| HSP60 (Heat | Chaperone intracellular | Yes, virion | Enhances 3′ |
| shock protein 60) | protein produced in | | processing and strand |
| | response to intracellular | | transfer in HIV-1 DNA |
| | stress. Found on | | integration. |
| | chromosome 2 location | | |
| | 2q33.1. A member of | | |
| | the chaperonin family. A | | |
| | mitochondrial protein | | |
| | that may function as a | | |
| | signaling molecule in the | | |
| | innate immune system. | | |
| | Essential for folding and | | |
| | assembly of newly | | |
| | imported proteins into | | |
| | the mitochondria. Two | | |
| | transcript variants | | |
| | encoding the same | | |
| | protein have been | | |
| | identified for this gene. | | |
| HSP70 (Heat | Chaperone intracellular | Yes, virion | May bind HIV-1 gag |
| shock protein 70) | protein produced in | | polyprotein chain and |
| | response to intracellular | | maintain proper tertiary |
| | stress. Found on | | confirmation during |
| | chromosome 19 location | | intracellular transport |
| | 19q13.42. Binds to and | | from nucleus to plasma |
| | regulates Hsp70 activity. | | membrane. May |
| | The carboxyl terminus of | | participate in early |
| | Hsp70-interacting | | events in infection. |
| | protein (CHIP) is an | | Might participate in |
| | Hsp70-associated | | uncoating the viral |
| | ubiquitin ligase which | | capsid. May target |
| | ubiquitinates misfolded | | HIV-1 PIC to the |
| | proteins associated with | | nucleus. |
| | cytoplasmic chaperones. | | |
| HSC70 (also | Chaperone, heat shock | Yes, virion | May bind HIV-1 gag |
| called Hsp73) | protein, works with | | polyprotein chain and |
| | auxilin to remove clathrin | | maintain proper tertiary |
| | coated vesicles. Found | | confirmation during |
| | on chromosome 11 | | intracellular transport |
| | location 11q24.1. Heat- | | from nucleus to plasma |
| | inducible and | | membrane. May |
| | constitutively expressed | | participate in early |
| | proteins identified. | | events in infection. |
| | Binds to nascent | | Might participate in |
| | polypeptides to facilitate | | uncoating the viral |
| | correct folding. | | capsid. May target |
| | Functions as an ATPase | | HIV-1 PIC to the |
| | in the disassembly of | | nucleus. |
| | clathrin-coated vesicles | | |
| | during transport of | | |
| | membrane components | | |
| | through the cell. Two | | |
| | alternatively spliced | | |
| | variants have been | | |
| | characterized. | | |
| CypA | Immunophilin, peptidyl- | Yes, virion | Incorporated as a |
| (Cyclophilin A) | prolyl isomerase. Found | | component of the Gag |
| | on chromosome 7 | | molecule at a 1/10 |
| | location 7p13. | | ratio. Also interacts |
| | Catalyzes the cis-trans | | with Vpr, Vif, MA, Nef |
| | isomerization of proline | | and gp120env. Binds |
| | imidic peptide bonds in | | to the central region of |
| | oligopeptides, | | the CA protein |
| | accelerates the folding of | | (residues 85 to 93). |
| | proteins. | | Catalyzes the cis/trans |
| | | | isomerization of the |
| | | | Gly-89-Pro-90 peptide |
| | | | bond. The capsid |
| | | | sequence 87His-Ala- |
| | | | Gly-Pro-Ile-Ala92 |
| | | | (87HAGPIA92) |
| | | | encompasses the |
| | | | primary cyclophilin A |
| | | | binding site. Inhibits |
| | | | Itk (Interleukin-2 |
| | | | tyrosine kinase) |
| | | | catalytic activity, a |
| | | | cytoplasmic non- |
| | | | receptor protein |
| | | | tyrosine kinase of the |
| | | | Tec (Molecular class: |
| | | | tyrosine kinase, |
| | | | Molecular Function: |
| | | | protein-tyrosine kinase |
| | | | activity, Biological |
| | | | Process: cell |
| | | | communication, signal |
| | | | transduction) family |
| | | | that participates in the |
| | | | intracellular signaling |
| | | | events leading to T cell |
| | | | activation. A proline- |
| | | | dependent |
| | | | conformational switch |
| | | | within the Itk SH2 |
| | | | domain regulates |
| | | | substrate recognition |
| | | | and mediates |
| | | | regulatory interactions |
| | | | with the active site of |
| | | | CypA. Regulates the |
| | | | cis/trans |
| | | | interconversion of the |
| | | | imidic bond within the |
| | | | conserved proline |
| | | | residues of Vpr in vivo. |
| | | | Implicated in capsid |
| | | | final assembly and |
| | | | defense of HIV-1 |
| | | | against innate host |
| | | | restriction factors |
| | | | specifically Ref-1. |
| | | | CypA/CD147 (Type I |
| | | | integral membrane |
| | | | glycoprotein found on |
| | | | hemopoietic, |
| | | | microglial, endothelial |
| | | | and peripheral blood |
| | | | cells) interaction |
| | | | follows CypA |
| | | | interaction with surface |
| | | | heparins. Facilitates |
| | | | viral/host cell binding |
| | | | prior to gp120/CD4 |
| | | | and gp120/CXCR4 or |
| | | | CCR5 co-receptor |
| | | | interaction. Increases |
| | | | probability of |
| | | | successful infection |
| | | | when a small amount |
| | | | of virus has been |
| | | | transmitted. |
| FKBP12 (FK506 | A peptidyl prolyl | Yes, virion | Growth of chronically |
| binding proteins) | isomerase. Found on | | infected HIV-1 cells |
| | chromosome 20 location | | dependent on FKBP12 |
| | 20p13. There is | | in vitro. |
| | evidence of multiple | | |
| | alternatively spliced | | |
| | transcript variants for | | |
| | this gene. | | |
| Tsg101 (Tumor | Vesicular transport | Yes, virion | Helix-1 of p6 binds to |
| specific gene) | protein, a component of | | the binding groove in |
| (VPS28 | the endosomal sorting | | Tsg101. The PTAP |
| homolog) | complex known as | | motif of p6 binds |
| | ESCRT-I which activates | | Tsg101 linking the |
| | formation of ESCRT-II, | | efferent virion to the |
| | which in turn recruits | | host endosomal |
| | ESCRT-III, all | | process. AIP1 |
| | components of the multi | | interacts with Tsg101 |
| | vesicular body (MVB) | | and p6 forming a |
| | formation process. | | ternary complex that |
| | | | recruits CHMP |
| | | | (charges multi |
| | | | vesicular body |
| | | | proteins) proteins |
| | | | leading to the |
| | | | endosomal cascade |
| | | | culminating in viral |
| | | | fission. The proline |
| | | | rich motif in p6 mimics |
| | | | the adapter protein Hrs |
| | | | (hepatocyte growth |
| | | | factor-regulated |
| | | | tyrosine kinase |
| | | | substrate), a |
| | | | component of the |
| | | | MVB. |
| Tal (Tsg101- | Vesicular transport | Yes, virion | Regulates a Tsg101- |
| associated ligase | protein. Found on | | associated complex |
| also known as | chromosome 9 location | | responsible for the |
| LRSAM1/leucine | 9q33.3-q34.11. | | sorting of cargo into |
| rich repeat and | Regulates vesicular | | cytoplasm-containing |
| sterile alpha | trafficking processes in | | vesicles that bud at |
| motif containing | mammals. RING (really | | multi vesicular bodies |
| 1) | interesting new gene) | | and at the plasma |
| | finger necessary for | | membrane. The |
| | multiple | | ubiquitin E2 variant |
| | monoubiquitination of | | (UEV region) of |
| | Tsg101. Bivalent | | Tsg101 binds to the |
| | binding of Tsg101 to a | | PTAP-PSAP motif of |
| | PTAP motif and to a | | Tal near the COOH |
| | central region of Tal is | | terminus. Monomeric |
| | essential for Tal- | | ubiquitin binds to |
| | mediated ubiquitination | | Tsg101 at a site |
| | of Tsg101. RING finger | | outside the UEV. The |
| | motifs are found in | | coiled coil (CC) region |
| | proteins of regulatory | | of Tal interacts with the |
| | function linked to | | steadiness box (SB) of |
| | ubiquitin proteasome | | Tsg101. Tal mediated |
| | system. Promotes | | ubiquitination of |
| | ubiquitination of target | | Tsg101 inactivates |
| | proteins that have been | | sorting/endosomal |
| | recognized by the E3 | | directed function of |
| | enzymes. RING | | Tsg101 and |
| | functions as a scaffold | | translocates Tsg101 |
| | positioning the substrate | | from the plasma |
| | and the E2 enzyme | | membrane. Enzymes |
| | facilitating ubiquitin | | that remove the |
| | transfer. RING finger | | ubiquitin on Tsg101 |
| | E3s perform three | | work in concert with |
| | functions: (1) | | Tal. The coordinated |
| | recognition and binding | | activity of Tal and a Tal |
| | of the substrate, (2) | | specific |
| | recognition and binding | | deubiquitination |
| | of the E2 enzyme, and | | enzyme control the |
| | (3) transfer ubiquitin. | | recycling of the Tsg101 |
| | | | and the reloading of |
| | | | Tsg101 mediated |
| | | | cargo. Ubiquitination |
| | | | of Tsg101 may allow |
| | | | transient dissociation |
| | | | of Tsg101 from its |
| | | | cargo allowing for the |
| | | | next step in the |
| | | | ESCRT complex to |
| | | | assimilate the cargo of |
| | | | Tsg101 facilitating the |
| | | | endosomal pathway |
| | | | hijacked by the HIV |
| | | | virus. Therefore Tal |
| | | | accelerates the |
| | | | endosomal/sorting |
| | | | function of Tsg101. |
| AIP1/ALIX (ALG- | Vesicular transport | Yes, virion | The p6 domain of HIV- |
| 2-interacting | protein. Found on | | 1 forms a ternary |
| protein X [also | chromosome 3 location | | complex with AIP1 and |
| known as | 3p22.3. Functions in | | Tsg101 which recruit |
| PDCD61P | actin-dependent | | CHMP proteins directly |
| programmed cell | intracellular positioning | | via AIP1 and indirectly |
| death 6 | of endosomes. Interacts | | via ESCRT-II to form |
| interacting | with an EF-hand type | | ESCRT-III. p6 adopt a |
| protein]) | Ca²⁺-binding protein, | | helix-flexible helix |
| | ALG-2 (apoptosis-linked | | structure; a short helix- |
| | gene 2), through its C- | | 1 (amino acids 14-18) |
| | terminal proline-rich | | is connected to a |
| | region. CHMP4a and | | pronounced helix-2 |
| | CHMP4b (chromatin- | | (amino acids 33-44) by |
| | modifying protein; | | a flexible hinge region. |
| | charged multi vesicular | | Helix-2 binds to |
| | body protein), are also | | AIP1/ALIX, the site of |
| | binding partners. | | interaction with Vpr. |
| | CHMP4b and Alix | | This indicates that the |
| | participate in formation | | Vpr binding region of |
| | of multivesicular bodies | | p6 adopts different |
| | by cooperating with | | three dimension |
| | SKD1 (suppressor of K+ | | structures dependent |
| | transport defect 1), a | | on the viral life cycle |
| | dominant negative form | | context. |
| | of the AAA type ATPase. | | |
| | Involved in calcium- | | |
| | dependent apoptosis. | | |
| | Links early endosomal | | |
| | complexes | | |
| | (TSG101/ESCRT-I) and | | |
| | late endosomal | | |
| | complexes | | |
| | (CHMP4/ESCRT-III). | | |
| VPS4B (vacuolar | Vesicular transport | Yes, virion | Binds to the COOH |
| protein sorting 4 | protein. Member of the | | terminal half of |
| homolog B) | AAA protein family | | Tsg101, specifically |
| | (ATPases). Found on | | residues 330-377. |
| | chromosome 18 location | | Required for HIV |
| | 18q21-32-q21.33. | | fission. Approximately |
| | | | 5-25 VPS28 molecules |
| | | | incorporated into each |
| | | | virion. |
| APOBEC3G | Member of the cytidine | Yes, virion | Binds to accessory |
| | deaminase gene family, | | protein Vif. Vif reduces |
| | Induces viral DNA hyper | | viral incorporation and |
| | mutations converting a | | cellular expression of |
| | cytosine residue into a | | protein limiting viral |
| | uracil residue. Uracil | | hyper mutation. |
| | residues in DNA tag the | | Incorporated into virion |
| | nucleic acid for nuclease | | between the two zinc |
| | destruction. Found on | | coordination motifs in |
| | chromosome 22 location | | Vif (amino acids 54-124). |
| | 22q13.1-q13.2 | | Also binds to |
| | | | nucleocapsid |
| | | | sequence (amino acids |
| | | | 104-156). |
| APOBEC3F | Member of the cytidine | Yes, virion | Targeted by the COOH |
| | deaminase gene family. | | terminus of Vif |
| | Functionally related to | | accessory protein |
| | the C to U RNA-editing | | limiting viral DNA |
| | cytidine deaminase | | hyper mutation. |
| | APOBEC1. Controls | | Localization by Vif to |
| | degradation of cell cycle | | processing or P-bodies |
| | proteins. Found on | | (cytoplasmic |
| | chromosome 22 location | | compartments involved |
| | 22q13.1. | | in the degradation and |
| | | | storage of non |
| | | | translating mRNAs). |
| | | | Induces G to A hyper |
| | | | mutations in newly |
| | | | synthesized minus |
| | | | strand viral cDNA at |
| | | | the step of reverse |
| | | | transcription. |
| UNG (Uracil- | Uracil-DNA glycosylase | Yes, virion | Integrase is required |
| DNA | removes DNA uracil | | for packaging of UNG |
| glycosylase) | residues. Excises the | | into virions. UNG2 |
| | uracil residues and | | binds the viral reverse |
| | introduces non | | transcriptase enzyme. |
| | templated nucleotides | | Uracil repair pathway |
| | allowing for somatic | | is associated with HIV- |
| | hyper mutation. | | 1 viral particles. |
| | Increases | | |
| | immunoglobulin | | |
| | diversity. Essential for | | |
| | generation of strand | | |
| | breaks for class switch | | |
| | recombination. Both | | |
| | mitochondrial (UNG1) | | |
| | and nuclear (UNG2) | | |
| | isoforms have been | | |
| | described. UNG1 only | | |
| | uracil-DNA glycosylase | | |
| | isolated to date in | | |
| | mitochondria. | | |
| | Mitochondrial UNG1 is | | |
| | encoded by nuclear not | | |
| | mitochondrial DNA. | | |
| | UNG2 predominant form | | |
| | in proliferating cells, | | |
| | UNG1 predominant form | | |
| | in non-proliferating cells. | | |
| | UNG2 levels high in S- | | |
| | phase and early G2 of | | |
| | the cell cycle. UNG2 | | |
| | primarily located at | | |
| | replication foci during S- | | |
| | phase. A second uracil- | | |
| | DNA glycosylase, | | |
| | Single-strand-selective | | |
| | Monofunctional Uracil- | | |
| | DNA Glycosylase | | |
| | (SMUG1) has a | | |
| | preference for double- | | |
| | stranded DNA rather | | |
| | than single-stranded | | |
| | DNA as with UNG1 and | | |
| | UNG2. Found on | | |
| | chromosome 12 location | | |
| | 12q23-q24.1. Not cell | | |
| | cycle regulated, does not | | |
| | accumulate at replication | | |
| | fosi and is not found in | | |
| | mitochondria. SMUG1 | | |
| | accumulates in nucleoli, | | |
| | UNG2 excluded from | | |
| | nucleoli. UNG1, UNG2 | | |
| | and SMUG1 function in | | |
| | base excision repair. | | |
| | UNG2 implicated in both | | |
| | innate and acquired | | |
| | immunity. | | |
| Staufen | Double-stranded RNA | Yes, virion | Binds HIV-1 genomic |
| | binding protein. | | RNA. May be involved |
| | Transports mRNAs to | | in retroviral genome |
| | intracellular | | selection and |
| | compartments/organelles. | | packaging into |
| | Found on | | assembling virions. |
| | chromosome 20 location | | Interaction with the |
| | 20q13.1. Binds tubulin. | | nucleocapsid domain |
| | Transports mRNA via | | of pr55(Gag) in vitro |
| | the microtubule network | | and in live cells |
| | to the RER. Five | | mediated by Staufen's |
| | transcript variants from | | dsRBD3 (RNA binding |
| | alternative splicing of | | domain 3), with a |
| | STAU gene encoding | | contribution from its C- |
| | three isoforms have | | terminal domain. |
| | been described. | | Preferentially binds |
| | | | with the 9-kb non- |
| | | | spliced viral RNA. |
| | | | Implicated in the |
| | | | generation of infectious |
| | | | virions. |
| INI1 (Integrase | INI1 is one of four | Yes, virion | INI1 has two highly |
| interactor | components of the | | conserved domains |
| 1)/hSNF5 | mammalian SWI (yeast | | known as imperfect |
| | mating type switch | | repeats (Rpt1 and |
| | gene)/SNF (sucrose non | | Rpt2). HIV-1 integrase |
| | fermentable gene) | | specifically binds to |
| | complex involved in | | Rpt 1, not Rpt 2. INI1 |
| | ATP-dependent | | is incorporated into the |
| | chromosome | | virions and is |
| | remodeling. Found on | | necessary for viral |
| | chromosome 22 | | production. Couples |
| | locations 22q11.23 and | | DNA binding of HIV-1 |
| | 22q11. | | PIC to the host cell |
| | | | transcriptional |
| | | | apparatus. Possibly |
| | | | couples integration and |
| | | | transcription. |
| | | | Stimulates the |
| | | | integration protein of |
| | | | the transcription |
| | | | coactivator PC4 |
| | | | (LEDGF/p75). |
| EF-1α | Catalyzes the entry of | Yes, | Interacts with MA (p17) |
| | aminoacyl-tRNA into the | associated | and the nucleocapsid |
| | ribosomal A (aminoacyl | with MA and | protein (p7). Basic |
| | site). Energy source | NC | residues in MA and NC |
| | hydrolysis of GTP. | | and possibly viral RNA |
| | Forms ternary complex | | are required for |
| | with GTP and all | | binding. May allow |
| | aminoacyl-tRNAs except | | RNA to be packaged |
| | the initiator tRNAᵢ^{met}. | | into virions. Catalyzes |
| | Isolated as an actin- | | the binding of RNA |
| | binding and bundling | | Polymerase II and |
| | protein in some cell | | TRP-185 to HIV-1 TAR |
| | lines. May control cell | | RNA. May interact |
| | cycle progression via its | | with Tat. |
| | actin interaction. Found | | |
| | on chromosome 6 | | |
| | location 6q14.1. Multiple | | |
| | copies found on other | | |
| | chromosomes | | |
| | representing pseudo | | |
| | genes have been | | |
| | defined. | | |
| LEDGF/p75 | DNA-binding protein | Yes, PIC | Central core domain |
| (lens epithelium- | implicated in cellular | (preintegration | and N-terminal zinc |
| derived growth | differentiation and | complex) | binding domain of |
| factor/transcription | cellular response to | | integrase are involved |
| coactivator p75 | environmental stress. | | in the interaction with |
| [alternate names | Activates transcription of | | LEDGF/p75. An |
| include | stress related genes | | essential cofactor for |
| PC(positive cofactor) | triggering a survival | | nuclear targeting of |
| 4 and | response. Protective | | HIV-1 integrase. |
| SFRS1 | role in stress-induced | | Physically links |
| interaction | apoptosis. Found on | | integrase to host |
| protein 2 | chromosome 9 location | | chromatin. The |
| (PSIP2)]) Prior | 9p22.3. A member of | | alternatively spliced |
| journal articles | the hepatoma-derived | | protein LEDGF/p52, |
| differentiate p75 | growth factor (HDGF). | | does not interact with |
| from PC4 in | The alternatively spliced | | HIV-1 or HIV-2 |
| HELA cells | p52 (PC4 and SFRS1 | | integrase. LEDGF/p75 |
| | interaction protein 1 | | links the integrase |
| | (PSIP1)) protein | | protein to the host |
| | interactions with | | chromatin during the |
| | transcriptional | | G₂ phase of the cell |
| | coactivators, general | | cycle. May target the |
| | transcription factors, and | | HIV-1 proviral DNA to |
| | splicing factors, | | specific genomic sites |
| | modulating pre-mRNA | | of actively transcribed |
| | splicing of class II genes. | | genes to promote viral |
| | The p75 protein is not a | | transcription. |
| | transcriptional factor. | | Residues are the |
| | Heparin binds to | | integrase binding |
| | LEDGF/p75, facilitating | | domain (IBD). Dictates |
| | transport through the | | site(s) of HIV |
| | cytoplasm into the | | integration, most |
| | nucleus. The N-terminal | | favored are areas |
| | PWWP domain and its | | undergoing |
| | beta-barrel substructure | | transcription, high G-C |
| | are needed for binding to | | (guanine-cytosine) |
| | metaphase chromatin. | | content, with active |
| | | | RNA polymerase |
| | | | subunits and |
| | | | transcription factors. |
| | | | Prevents proteasomal |
| | | | degradation of HIV-1 |
| | | | integrase. The N- |
| | | | terminal zinc binding |
| | | | domain (amino acids |
| | | | 1-52) and the central |
| | | | core domain (amino |
| | | | acids 53-212) of HIV-1 |
| | | | integrase interact with |
| | | | LEDGF/p75. The core |
| | | | domain harbors the |
| | | | main determinant for |
| | | | this interaction. |
| DNA-PK (DNA- | Repairs double-stranded | Yes, virion, | Participates in |
| dependent | DNA breaks by | PIC | retroviral DNA |
| protein kinase) | nonhomologous and | | integration. Sp1 is a |
| | joining (NHEJ). | | mammalian |
| | Composed of DNA | | transcription factor |
| | binding protein, | | characterized by |
| | Ku70/Ku86 heterodimer | | clusters of zinc fingers. |
| | and a large catalytic | | Zinc fingers are |
| | subunit, DNA-PK_{cs} (a | | essential for correct |
| | serine/threonine protein | | folding and DNA |
| | kinase). The DNA-PK_{cs} | | binding. Tat amino |
| | is found on chromosome | | acids 30 to 55 binds to |
| | 8 location 8q11. | | the transcription factor |
| | Participates in | | Sp1. Tat augments |
| | variable/diversity/joining | | double-stranded DNA- |
| | recombination events in | | PK-mediated Sp1 |
| | T and B cells. The | | phosphorylation in a |
| | PRKDC gene encodes | | contact-dependent |
| | the catalytic subunit | | manner. |
| | DNA-PK_{cs}. The DNA- | | |
| | binding component is the | | |
| | autoimmune antigen Ku | | |
| | (MIM 152690). On its | | |
| | own, the catalytic | | |
| | subunit of DNA-PK is | | |
| | inactive and relies on the | | |
| | Ku component for | | |
| | nuclear localization and | | |
| | kinase activity. DNA- | | |
| | PK_{cs} is only function | | |
| | when bound to DNA. | | |
| Ku80 | 86-kDa subunit of | Yes, virion, | Required for the |
| | human DNA-dependent | PIC | formation of the |
| | protein kinase. Ku80 | | retroviral 2-LTR circle |
| | protein forms | | DNA. Protects |
| | heterodimer with the | | infected cells from |
| | Ku70 subunit to form a | | retrovirus-induced |
| | complex that possesses | | apoptosis. Binds to |
| | a DNA end-binding | | viral cDNA. Enhances |
| | activity. Ku70/Ku80 | | binding of reverse |
| | heterodimer can recruit | | transcriptase and/or |
| | the catalytic p350 | | integrase as well as |
| | subunit of the DNA- | | the PIC associated |
| | dependent protein | | host cellular cofactors. |
| | kinase. Catalyzes DNA | | The DNA repair |
| | double-strand break | | pathway is another |
| | repair (nonhomologous | | cellular process |
| | recombinational | | hijacked by HIV to |
| | repair/NHEJ). Found on | | complete life cycle. |
| | chromosome 2 location | | |
| | 2q35. Implicated in | | |
| | transcription, | | |
| | variable/diversity/joining | | |
| | gene recombination in T | | |
| | and B cells and telomere | | |
| | maintenance. | | |
| hRad18 | The protein encoded by | Yes, virion, | hRAD18 possesses a |
| | this gene is similar to the | PIC | RING (Really |
| | S. cerevisiae DNA | | Interesting New Gene) |
| | damage repair protein | | finger domain, a |
| | Rad18. Yeast Rad18 | | structure associated |
| | interacts with Rad6, an | | with E3 ubiquitin |
| | ubiquitin-conjugating | | ligases. A 162-residue |
| | enzyme (E2) required for | | region of hRad18 |
| | post-replication repair of | | (amino acids 65-226) |
| | damaged DNA. Similar | | binds with and |
| | to its yeast counterpart, | | stabilizes integrase. |
| | hRad18 interacts with | | Integrase is inherently |
| | the human homolog of | | unstable since its N |
| | yeast Rad6 protein | | terminal amino acid is |
| | through a conserved | | phenylalanine. N |
| | ring-finger motif. Found | | terminal phenylalanine |
| | on chromosome 3 | | is recognized as a |
| | location 3p25-p24. | | degradation signal by |
| | | | the ubiquitin |
| | | | proteasome system |
| | | | (N-end Rule). hRad18 |
| | | | interacts with HIV-1 |
| | | | integrase in |
| | | | replication/translesion |
| | | | DNA repair in the |
| | | | retroviral integration |
| | | | process. |
| EED (Embryonic | Nuclear protein involved | Yes, virion (?) | Interacts with HIV-1 |
| ectoderm | in transcriptional | | matrix and integrase |
| development) | repression and gene | | early in the HIV viral |
| | silencing by histone de- | | life cycle. May |
| | acetylation. Found on | | facilitate replication. |
| | chromosome 11 location | | Nef translocates EED |
| | 11q14.2-q22.3. May | | from the nucleus to the |
| | regulate integrin | | plasma membrane. |
| | function. Two distinct | | This stimulates Tat- |
| | isoforms identified. A | | dependent HIV |
| | member of the | | transcription. |
| | superfamily of WD-40 | | |
| | repeat proteins and of | | |
| | the Polycomb group | | |
| | proteins. | | |
| HMGA1/HMG-1a | A non-histone protein, a | Yes, virion, | Increases HIV |
| (high mobility | general coactivator of | PIC | integrase activity by a |
| group AT-hook1) | transcription, involved in | | factor of 10. |
| | many cellular processes, | | |
| | including regulation of | | |
| | inducible gene | | |
| | transcription. | | |
| | Preferentially binds to | | |
| | the minor groove of A + T | | |
| | (adenine + thymine) rich | | |
| | regions in double- | | |
| | stranded DNA. | | |
| | Frequently acetylated | | |
| | and found in the | | |
| | nucleus. At least seven | | |
| | transcript variants | | |
| | encoding two different | | |
| | isoforms have been | | |
| | found for this gene. | | |
| | Found on chromosome 6 | | |
| | location 6p21. A | | |
| | characteristic feature is | | |
| | the ability to bend DNA. | | |
| BAF (barrier-to- | Interacts with nuclear | Yes, virion, | Increases HIV |
| auto integration | proteins that have a | PIC and in | integrase activity by |
| factor)/BANF1 | conserved LEM (LAP2 | intact virions | 5 × 10³. Prevents HIV-1 |
| | [lamin-associated | Approximately | cDNA auto integration. |
| | polypeptide 2], emerin, | zero to three | Promotes efficient |
| | MAN1) domain. | copies per | intermolecular |
| | Frequently found in DNA | virion. | recombination of viral |
| | polymerases, ligases, | | and host DNA. HIV-1 |
| | glycosylases and | | cDNA associated with |
| | helicases that bind DNA | | emerin in vivo, and the |
| | non-specifically. | | interaction of viral |
| | Definitive function | | cDNA with chromatin |
| | unknown. May reform | | was dependent on |
| | the nuclear envelope | | emerin. Required for |
| | during telophase (the | | the association of viral |
| | final stage of mitosis | | cDNA with emerin (an |
| | characterized by | | inner nuclear envelope |
| | cytokinesis or cell | | protein) supporting |
| | division). Found on | | viral replication. |
| | chromosome 11 location | | |
| | 11q13.1. | | |
| p300 | A generalized | Yes, PIC | Tat binds two p300 |
| | transcriptional co- | | and CBP both in vitro |
| | activator with histone | | and in vivo. Integrity of |
| | acetylase activity. | | the basic domain of |
| | Found on chromosome | | Tat is essential for this |
| | 22 location 22q13.2. | | interaction. HIV-1 Tat |
| | p300 is related by | | forms a ternary |
| | sequence to CBP | | complex with P/CAF |
| | (CREB-binding protein | | and p300. This |
| | [CREB: cyclic-AMP | | increases the affinity of |
| | responsive element | | p300 for CDK9/P-TEFb |
| | binding protein]). Like | | CTD kinase complex. |
| | CBP can stimulate | | Tat binds to amino acid |
| | transcription through | | 1253-1790 of p300. |
| | activation of CREB. | | This interaction results |
| | | | in a structural change |
| | | | of p300/CBP. Tat-p300 |
| | | | interaction increases |
| | | | the HAT activity of |
| | | | p300 on histone H4. |
| | | | H4 is a component of |
| | | | nucleosomes. Histone |
| | | | H4 was acetylated on |
| | | | lysines 8, 12, and 16. |
| | | | Acetylation of H4 was |
| | | | inhibited by Lys- |
| | | | coenzyme A (CoA), a |
| | | | selective inhibitor of |
| | | | p300 acetyltransferase |
| | | | activity. Tat could auto |
| | | | acetylate itself, which |
| | | | was specific to lysine |
| | | | residues 41 and 71. |
| | | | Acetylated Tat is |
| | | | considered to be the |
| | | | transcriptionally active |
| | | | form intracellularly. |
| | | | p300 and PCAF |
| | | | directly acetylate Tat. |
| | | | p300 acetylated Lys50 |
| | | | in the TAR RNA |
| | | | binding domain, while |
| | | | PCAF acetylated |
| | | | Lys28 in the activation |
| | | | domain of Tat. |
| | | | Acetylation at Lys28 by |
| | | | PCAF enhanced Tat |
| | | | binding to the Tat- |
| | | | associated kinase, |
| | | | CDK9/P-TEFb, while |
| | | | acetylation by p300 at |
| | | | Lys50 of Tat promoted |
| | | | the dissociation of Tat |
| | | | from TAR RNA. |
| | | | Acetylation of Tat |
| | | | regulates two discrete |
| | | | and functionally critical |
| | | | steps in viral |
| | | | transcription (1) |
| | | | binding to an RNAP II |
| | | | CTD-kinase, (2) |
| | | | release of Tat from |
| | | | TAR RNA. Vpr |
| | | | induced G2/M growth |
| | | | arrest is mediated by |
| | | | p300 which promotes |
| | | | cooperative |
| | | | interactions between |
| | | | the Rel A subunit of |
| | | | NF_{K}B and cyclin |
| | | | B1.Cdc2. Vpr interacts |
| | | | with p300 which |
| | | | controls in part intra |
| | | | cellular NF_{K}B activity. |
| | | | Therefore, Vpr controls |
| | | | in part HIV |
| | | | transcription via p300. |
| Rev cofactor | Related to nucleoporins | No? | hRIP is an essential |
| (RCF) (HRB | that mediate nuclear | | cellular Rev cofactor |
| [HIV-1 Rev | cytoplasmic transport. | | which functions at a |
| binding protein]), | Found on chromosome 2 | | step in HIV-1 RNA |
| (hRIP [human | location 2q36.3. | | export: movement of |
| Rev-interacting | | | mRNAs from the |
| protein]) | | | nucleus. Promotes the |
| | | | release of incompletely |
| | | | spliced HIV-1 RNAs |
| | | | from the perinuclear |
| | | | region. |
| HSP90 (Heat | HSPs, chaperone | No | Tat enters T cells toxin, |
| shock protein 90) | intracellular protein | | using clathrin-mediated |
| | produced in response to | | endocytosis before |
| | intracellular stress. | | low-pH-induced and |
| | Required for | | Hsp90-assisted |
| | translocation of FGF-1 | | endosomal |
| | and FGF-2 across the | | translocation. Critical |
| | endosomal membrane. | | to the |
| | Involved in types I and II | | stabilization/folding of |
| | interferon pathways. | | Cdk9 as well as the |
| | Found on chromosome | | assembly of an active |
| | 14 location 14q32.33. | | Cdk9/cyclin T1 |
| | | | complex responsible |
| | | | for P-TEFb-mediated |
| | | | Tat transactivation. |
| CypB | Immunophilin with cis- | No | Interacts with HIV-1 |
| (Cyclophilin B) | trans peptidyl-prolyl | | Gag polyprotein |
| | isomerase and | | Pr55gag. HIV-1 Gag |
| | chaperone-like activities. | | directly contacts |
| | Found on chromosome | | residues in the |
| | 15 location 15q21-q22. | | hydrophobic pocket of |
| | Primarily located within | | CyPA. Binds with |
| | the endoplasmic | | higher affinity to |
| | reticulum. Associated | | mature capsid protein |
| | with the secretory | | cleaved from the Gag |
| | pathway and release of | | polyprotein. |
| | biological fluids. | | |
| HSP27 | HSPs, chaperone. | No | May link the efferent |
| | Induced by thermal, free | | loop of the replication |
| | radical, and | | cycle to the endosomal |
| | inflammatory stress. | | pathway. |
| | Chaperone denatured | | |
| | intracellular proteins, | | |
| | signal transduction | | |
| | proteins, modulating | | |
| | signaling cascades | | |
| | during repeated stress. | | |
| | Found on chromosome 7 | | |
| | location 7q11.23. | | |
| HSP40 | HSPs, chaperone. | No | Interacts with Nef |
| | Found on chromosome | | which induces its |
| | 19 location 19p13.2. | | expression. Nef |
| | Induced by thermal, free | | translocates Hsp40 |
| | radical, and | | into the nucleus of |
| | inflammatory stress. | | infected cells. This |
| | Chaperone denatured | | facilitates viral gene |
| | intracellular proteins, | | expression. Becomes |
| | signal transduction | | part of the cyclin- |
| | proteins, modulating | | dependent kinase 9- |
| | signaling cascades | | associated |
| | during repeated stress. | | transcription complex |
| | | | regulating long |
| | | | terminal repeat- |
| | | | mediated gene |
| | | | expression. |
| VPS37B | Vesicular transport | No | Ternary complex with |
| | protein. Found on | | Tsg 101 and VPS 28 |
| | chromosome 12 location | | forms the human |
| | 12q24.31. Component | | ESCRT-I which is |
| | of the human ESCRT-I | | required for HIV-1 Gag |
| | complex. Forms a | | budding and virus |
| | complex with Ts101 and | | infectivity |
| | Vps28. | | |
| CD4 | A type I transmembrane | Yes, envelope | Interacts with specific |
| | protein found on | | domains of gp120 |
| | helper/inducer T cells, | | facilitating viral fusion. |
| | monocytes, | | |
| | macrophages, and | | |
| | dendritic cells that is | | |
| | involved in T-cell | | |
| | recognition of antigens. | | |
| | Found on chromosome | | |
| | 12 location 12pter-p12. | | |
| CXCR4 | Binds chemokine SDF-1 | Yes, envelope | Viral co-receptor |
| | (stromal cell derived | | determines viral |
| | factor 1). Found on | | tropism for CD4 T |
| | hematopoietic | | cells. |
| | precursors, mature white | | |
| | blood cells and plasma | | |
| | cells. Found on | | |
| | chromosome 2 location | | |
| | 2q21. Type III | | |
| | transmembrane protein | | |
| | crossing the plasma | | |
| | membrane seven times. | | |
| CCR5 | Found on Th1 cells, | Yes, envelope | Viral co-receptor |
| | dendritic cells, | | determines viral |
| | monocytes/macrophages. | | tropism for |
| | Type III | | macrophages. |
| | transmembrane protein | | |
| | crossing the plasma | | |
| | membrane seven times. | | |
| | Ligands include | | |
| | monocyte chemo | | |
| | attractant protein 2 | | |
| | (MCP-2), macrophage | | |
| | inflammatory protein 1 | | |
| | alpha (MIP-1 alpha), | | |
| | macrophage | | |
| | inflammatory protein 1 | | |
| | beta (MIP-1 beta) and | | |
| | regulated on activation | | |
| | normal T expressed and | | |
| | secreted protein | | |
| | (RANTES). Found on | | |
| | chromosome 3 location | | |
| | 3p21.31 | | |
| CD86 | Member of the | Yes, envelope | Skews the host |
| | immunoglobulin | | towards a Th2 biased |
| | superfamily. Membrane | | immune response. |
| | protein present on some | | |
| | germinal-center B cells, | | |
| | mitogen-activated B | | |
| | cells, and monocytes | | |
| | that serves as a B-cell | | |
| | activator. Found on | | |
| | chromosome 3 location | | |
| | 3q21. Co-stimulatory | | |
| | signal necessary for | | |
| | activation of T cell. | | |
| Phosphatidyl | Intermediate in the | No, virion? | Promotes binding of |
| inositol 4,5- | plasma membrane | | Gag to the plasma |
| bisphosphate | generation of inositol | | membrane to facilitate |
| [PI(4,5)P₂] | triphosphate (IP₃) and | | protein/protein |
| | diacylglycerol (DAG). | | interactions involving |
| | IP3 releases calcium | | the capsid domains. |
| | from the endoplasmic | | |
| | reticulum and DAG | | |
| | activates protein kinase | | |
| | C (PKC). Found on | | |
| | chromosome 22 location | | |
| | 22q11.2-q13.2. | | |
| NF_{K}B | Cellular transcription | | Binding sites in the |
| | factor involved in the | | viral LTR necessary for |
| | immune process. Found | | viral transcription. |
| | on chromosome * | | |
| | location *. | | |
| NFAT | Cellular transcription | | Binding sites in the |
| | factor involved in the | | viral LTR necessary for |
| | immune process. Found | | viral transcription. |
| | on chromosome 20 | | |
| | location 20q13.2-q13.3. | | |
| Sp1 | Cellular transcription | | Binding sites in the |
| | factor involved in the | | viral LTR necessary for |
| | immune process. Found | | viral transcription. |
| | on chromosome 12 | | |
| | location 12q13.1. | | |
| Cyclin T | | | |
| CDK9/PITALRE | | | |
| RNA polymerase | | | |
| II | | | |
| Exportin 1/Crm 1 | | | |
| Ran GTP | | | |
| Ran GTPase | | | |
| activating protein | | | |
| (RanGAP) | | | |
| Ran Binding | | | |
| Protein | | | |
| (RanBP1) | | | |
| CCR2B | | | |
| CCR3 | | | |
| CCR8 | | | |
| GPR1 | | | |
| GPR15 (Bob) | | | |
| STRL33 (Bonzo) | | | |
| US28 | | | |
| CX3CR1 (V28) | | | |
| APJ | | | |
| chemR23 | | | |
| Furin | | | |
| H-β-TrCP | | | |
| Skp1p | | | |
| AP-2 | | | |
| C4 binding | | | |
| protein (C4b | | | |
| protein) | | | |
| CD35 | | | |
| (Complement- | | | |
| receptor 1) | | | |
| CD21 | | | |
| (Complement- | | | |
| receptor 2) | | | |
| sCR1 | | | |
| (Complement- | | | |
| receptor 1) | | | |
| Cyclin T | | | |
| CDK9 | | | |

HIV-2

| | | | |
|---|---|---|---|
| HLA-DR | Antigen presentation, | Yes, envelope | Interacts with CD4 |
| | MHC class II directly | | glycoprotein on target |
| | presents peptide | | cells. Without |
| | antigens to CD4 T cells. | | associated antigen in |
| | Highly polymorphic. | | the peptide binding |
| | Heterodimer consisting | | cleft of HLA-DR and |
| | of an alpha (DRA) and a | | co-stimulating |
| | beta (DRB) chain, both | | molecular interactions, |
| | anchored in the | | CD4 cell will be |
| | membrane. Presents | | rendered anergic. |
| | peptides derived from | | HIV-1 Gag expression |
| | extracellular proteins by | | is able to induce HLA- |
| | antigen presenting cells, | | DR cell-surface |
| | B cells, dendritic cells | | localization in H78- |
| | and macrophages. | | C10.0 cells. In human |
| | Found on chromosome | | macrophages, HIV-1 |
| | 6 location 6p21.3. | | Gag proteins co- |
| | | | localize with MHC II |
| | | | (HLA-DR), CD63, and |
| | | | Lamp1 in MHC II |
| | | | compartments. HIV-1 |
| | | | Capsid (p24) inhibits |
| | | | interferon gamma |
| | | | induced increases in |
| | | | HLA-DR and |
| | | | cytochrome B heavy |
| | | | chain mRNA levels in |
| | | | the human monocyte- |
| | | | like cell line THP1. |
| | | | HIV-1 Tat down |
| | | | regulates expression |
| | | | of MHC class II genes |
| | | | in antigen-presenting |
| | | | cells (APC) by |
| | | | inhibiting the |
| | | | transactivator of MHC |
| | | | class II genes, CIITA. |
| | | | HIV-1 Tat up regulates |
| | | | HLA-DR expression in |
| | | | monocyte-derived |
| | | | dendritic cells and T |
| | | | cells, thereby driving T |
| | | | cell-mediated immune |
| | | | responses and |
| | | | activation. Associates |
| | | | with HIV-1 gp41. |
| | | | Enhances HIV-1 |
| | | | infectivity. Not |
| | | | affected by viral |
| | | | tropism which is |
| | | | determined by the V3 |
| | | | loop of gp120. Amino |
| | | | acids 708-750 of gp41 |
| | | | required for MHC-II |
| | | | incorporation into the |
| | | | HIV-1 envelope. |
| MHC-1 | In humans, six MHC | Yes, envelope | Enhances HIV |
| | class 1 isotypes have | | infectivity and changes |
| | been identified: HLA-A, | | gp120 conformation. |
| | HLA-B, HLA-C, HLA-E, | | Without antigen in |
| | HLA-F and HLA-G. | | MHC-1 binding groove |
| | HLA-A, HLA-B and HLA- | | and co-stimulatory |
| | C function to present | | activity, anergy results. |
| | antigens to CD8 T cells | | HIV-1 Nef down |
| | and to form ligands for | | regulates surface |
| | natural killer (NK) cell | | expression of CD4 and |
| | receptors. HLA-E and | | MHC-1 in resting CD4⁺ |
| | HLA-G also ligands for | | T lymphocytes. Nef |
| | NK-cell receptors. HLA- | | up regulates cell |
| | A is found on | | surface levels of the |
| | chromosome 6 location | | MHC-2 invariant chain |
| | 6p21.3. | | CD74. Nef down |
| | | | regulates HLA class I |
| | | | expression and |
| | | | therefore suppresses |
| | | | the cytolytic activity of |
| | | | HIV-1-specific |
| | | | cytotoxic T-lymphocyte |
| | | | (CTL) clones. |
| | | | Macrophage-tropic (M- |
| | | | tropic) HIV-1 Nef down |
| | | | regulates expression |
| | | | of HLA-A2 on the |
| | | | surface of productively |
| | | | infected macrophages. |
| | | | HIV-1 group N and |
| | | | group O Nef weakly |
| | | | down regulates CD4, |
| | | | CD28, and class I and |
| | | | II MHC molecules and |
| | | | up regulates surface |
| | | | expression of the |
| | | | invariant chain (Ii) |
| | | | associated with |
| | | | immature major |
| | | | histocompatibility |
| | | | complex (MHC) class |
| | | | II. Nef interrupts |
| | | | MHC-I trafficking to |
| | | | the plasma membrane |
| | | | and inhibits antigen |
| | | | presentation. Nef |
| | | | interacts with the μ1 |
| | | | subunit of adaptor |
| | | | protein (AP) AP-1A, a |
| | | | cellular protein |
| | | | complex implicated in |
| | | | TGN linking |
| | | | endosome/lysosome |
| | | | pathways. HIV-1 Nef |
| | | | binds to the MHC-I |
| | | | (HLA-A2) hypo |
| | | | phosphorylated |
| | | | cytoplasmic tails in the |
| | | | endoplasmic |
| | | | reticulum; this Nef- |
| | | | MHC-I complex |
| | | | migrates into the Golgi |
| | | | apparatus then into |
| | | | the lysosomal |
| | | | compartments for |
| | | | degradation. Nef |
| | | | promotes a physical |
| | | | interaction between |
| | | | endogenous AP-1 and |
| | | | MHC-I. The Pro-X-X- |
| | | | Pro motif in HIV-1 Nef |
| | | | induces the |
| | | | accumulation of CCR5 |
| | | | (HIV-1 M-tropic |
| | | | coreceptor) in a |
| | | | perinuclear |
| | | | compartment where |
| | | | both molecules co- |
| | | | localize with MHC-1. |
| | | | The Pro-X-X-Pro motif |
| | | | interacts with src |
| | | | homology region-3 |
| | | | domains of src-like |
| | | | kinases interfering with |
| | | | cell signaling |
| | | | pathways. HIV-1 Nef |
| | | | selectively down |
| | | | regulates HLA-A and |
| | | | HLA-B but does not |
| | | | significantly affect |
| | | | HLA-C or HLA-E, |
| | | | which allows HIV- |
| | | | infected cells to avoid |
| | | | NK cell-mediated lysis. |
| | | | Nef decreases the |
| | | | incorporation of MHC- |
| | | | 1 molecules into |
| | | | virions. Furthermore, |
| | | | Nef down regulates |
| | | | MHC-1 expression on |
| | | | human dendritic cells. |
| | | | Therefore, HIV-1 Nef |
| | | | impairs antigen |
| | | | presentation to HIV- |
| | | | specific CD8+ T |
| | | | lymphocytes. HIV-1 |
| | | | Nef-induced down |
| | | | regulation of MHC-I |
| | | | expression and MHC-I |
| | | | targeting to the trans- |
| | | | Golgi network (TGN) |
| | | | require the binding of |
| | | | Nef to PACS-1 |
| | | | (phosphofurin acidic |
| | | | cluster sorting protein |
| | | | 1). PACS-1 is a |
| | | | protein with a putative |
| | | | role in the localization |
| | | | of proteins to the |
| | | | trans-Golgi network |
| | | | (TGN) including furin |
| | | | which cleaves gp160. |
| | | | HIV-1 Nef down |
| | | | regulates MHC-1 on |
| | | | lymphoid, monocytic |
| | | | and epithelial cells. |
| | | | Nef expression results |
| | | | in rapid internalization |
| | | | and accumulation of |
| | | | MHC-1 in endosomal |
| | | | vesicles which |
| | | | degrade MHC-1 |
| | | | molecules. Nef blocks |
| | | | transport of MHC-I |
| | | | molecules to the cell |
| | | | surface, leading to |
| | | | accumulation of MHC- |
| | | | 1 in intracellular |
| | | | organelles. |
| | | | Furthermore, the effect |
| | | | of Nef on MHC-1 |
| | | | molecules (but not on |
| | | | CD4) requires |
| | | | phosphoinositide 3- |
| | | | kinase (PI 3-kinase) |
| | | | activity found on the |
| | | | cytoplasmic side of the |
| | | | plasma membrane. |
| HSP70 (Heat | Chaperone intracellular | Yes, virion | May bind HIV-1 gag |
| shock protein | protein produced in | | polyprotein chain and |
| 70) | response to intracellular | | maintain proper |
| | stress. Found on | | tertiary confirmation |
| | chromosome 19 location | | during intracellular |
| | 19q13.42. Binds to and | | transport from nucleus |
| | regulates Hsp70 activity. | | to plasma membrane. |
| | The carboxyl terminus of | | May participate in |
| | Hsp70-interacting | | early events in |
| | protein (CHIP) is an | | infection. Might |
| | Hsp70-associated | | participate in |
| | ubiquitin ligase which | | uncoating the viral |
| | ubiquitinates misfolded | | capsid. May target |
| | proteins associated with | | HIV-1 PIC to the |
| | cytoplasmic | | nucleus. |
| | chaperones. | | |
| UNG (Uracil- | Uracil-DNA glycosylase | Yes, virion | Integrase is required |
| DNA | removes DNA uracil | | for packaging of UNG |
| glycosylase) | residues. Excises the | | into virions. UNG2 |
| | uracil residues and | | binds the viral reverse |
| | introduces non | | transcriptase enzyme. |
| | templated nucleotides | | Uracil repair pathway |
| | allowing for somatic | | is associated with HIV- |
| | hyper mutation. | | 1 viral particles. |
| | Increases | | |
| | immunoglobulin | | |
| | diversity. Essential for | | |
| | generation of strand | | |
| | breaks for class switch | | |
| | recombination. Both | | |
| | mitochondrial (UNG1) | | |
| | and nuclear (UNG2) | | |
| | isoforms have been | | |
| | described. UNG1 only | | |
| | uracil-DNA glycosylase | | |
| | isolated to date in | | |
| | mitochondria. | | |
| | Mitochondrial UNG1 is | | |
| | encoded by nuclear not | | |
| | mitochondrial DNA. | | |
| | UNG2 predominant form | | |
| | in proliferating cells, | | |
| | UNG1 predominant form | | |
| | in non-proliferating cells. | | |
| | UNG2 levels high in S- | | |
| | phase and early G2 of | | |
| | the cell cycle. UNG2 | | |
| | primarily located at | | |
| | replication foci during S- | | |
| | phase. A second uracil- | | |
| | DNA glycosylase, | | |
| | Single-strand-selective | | |
| | Monofunctional Uracil- | | |
| | DNA Glycosylase | | |
| | (SMUG1) has a | | |
| | preference for double- | | |
| | stranded DNA rather | | |
| | than single-stranded | | |
| | DNA as with UNG1 and | | |
| | UNG2. Found on | | |
| | chromosome 12 location | | |
| | 12q23-q24.1. Not cell | | |
| | cycle regulated, does | | |
| | not accumulate at | | |
| | replication fosi and is | | |
| | not found in | | |
| | mitochondria. SMUG1 | | |
| | accumulates in nucleoli, | | |
| | UNG2 excluded from | | |
| | nucleoli. UNG1, UNG2 | | |
| | and SMUG1 function in | | |
| | base excision repair. | | |
| | UNG2 implicated in both | | |
| | innate and acquired | | |
| | immunity. | | |
| Staufen | Double-stranded RNA | Yes, virion | Binds HIV-1 genomic |
| | binding protein. | | RNA. May be involved |
| | Transports mRNAs to | | in retroviral genome |
| | intracellular | | selection and |
| | compartments/organelles. | | packaging into |
| | Found on | | assembling virions. |
| | chromosome 20 location | | Interaction with the |
| | 20q13.1. Binds tubulin. | | nucleocapsid domain |
| | Transports mRNA via | | of pr55(Gag) in vitro |
| | the microtubule network | | and in live cells |
| | to the RER. Five | | mediated by Staufen's |
| | transcript variants from | | dsRBD3 (RNA binding |
| | alternative splicing of | | domain 3), with a |
| | STAU gene encoding | | contribution from its C- |
| | three isoforms have | | terminal domain. |
| | been described. | | Preferentially binds |
| | | | with the 9-kb non- |
| | | | spliced viral RNA. |
| | | | Implicated in the |
| | | | generation of |
| | | | infectious virions. |
| α-actinin 1 | | | Required for Vpx- |
| | | | mediated nuclear |
| | | | import of the PIC. |
| LEDGF/p75 | DNA-binding protein | Yes, PIC | Central core domain |
| (lens epithelium- | implicated in cellular | (preintegration | and N-terminal zinc |
| derived growth | differentiation and | complex) | binding domain of |
| factor/transcription | cellular response to | | integrase are involved |
| coactivator | environmental stress. | | in the interaction with |
| p75 [alternate | Activates transcription of | | LEDGF/p75. An |
| names include | stress related genes | | essential cofactor for |
| PC(positive cofactor) | triggering a survival | | nuclear targeting of |
| 4 and | response. Protective | | HIV-1 integrase. |
| SFRS1 | role in stress-induced | | Physically links |
| interaction | apoptosis. Found on | | integrase to host |
| protein 2 | chromosome 9 location | | chromatin. The |
| (PSIP2)]) Prior | 9p22.3. A member of | | alternatively spliced |
| journal articles | the hepatoma-derived | | protein LEDGF/p52, |
| differentiate p75 | growth factor (HDGF). | | does not interact with |
| from PC4 in | The alternatively spliced | | HIV-1 or HIV-2 |
| HELA cells | p52 (PC4 and SFRS1 | | integrase. |
| | interaction protein 1 | | LEDGF/p75 links the |
| | (PSIP1)) protein | | integrase protein to |
| | interactions with | | the host chromatin |
| | transcriptional | | during the G₂ phase of |
| | coactivators, general | | the cell cycle. May |
| | transcription factors, and | | target the HIV-1 |
| | splicing factors, | | proviral DNA to |
| | modulating pre-mRNA | | specific genomic sites |
| | splicing of class II | | of actively transcribed |
| | genes. The p75 protein | | genes to promote viral |
| | is not a transcriptional | | transcription. |
| | factor. Heparin binds to | | Residues are the |
| | LEDGF/p75, facilitating | | integrase binding |
| | transport through the | | domain (IBD). |
| | cytoplasm into the | | Dictates site(s) of HIV |
| | nucleus. The N-terminal | | integration, most |
| | PWWP domain and its | | favored are areas |
| | beta-barrel substructure | | undergoing |
| | are needed for binding | | transcription, high G-C |
| | to metaphase | | (guanine-cytosine) |
| | chromatin. | | content, with active |
| | | | RNA polymerase |
| | | | subunits and |
| | | | transcription factors. |
| | | | Prevents proteasomal |
| | | | degradation of HIV-1 |
| | | | integrase. The N- |
| | | | terminal zinc binding |
| | | | domain (amino acids |
| | | | 1-52) and the central |
| | | | core domain (amino |
| | | | acids 53-212) of HIV-1 |
| | | | integrase interact with |
| | | | LEDGF/p75. The core |
| | | | domain harbors the |
| | | | main determinant for |
| | | | this interaction. |
| tRNA synthetase | Ligase, charges or | Yes, virion | tRNA^{lys3} binds to the |
| or aminoacyl | aminoacylates key RNA | | primer binding site |
| tRNA synthetase | molecules linking the | | initiating reverse |
| | molecule to the | | transcription. In HIV-1 |
| | respective amino acid. | | an RNA loop formed |
| | One synthetase for each | | by the tRNA^{lys3} |
| | amino acid found in | | anticodon and an |
| | mammalian cells. ATP | | adenine rich RNA loop |
| | dependent. | | initiates reverse |
| | | | transcription. |
| tRNA^{lys} | Allows incorporation of | Yes, virion | Induces three |
| | lysine into proteins by | associated | dimensional structural |
| | the host translational | attached to | changes in the |
| | apparatus. | primer binding | unspliced viral RNA to |
| | | site (PBS) | allow reverse |
| | | | transcription to |
| | | | proceed. |
| GAPDH | In glycolysis, | Yes, virion | ?????? |
| (Glyceraldehyde- | enzymatically converts | | |
| 3-phosphate | Glyceraldehyde-3- | | |
| dehydrogenase) | phosphate to 1,3- | | |
| | bisphosphoglycerate. | | |
| | Also involved in cell | | |
| | cycle regulation by | | |
| | modulating cyclin B- | | |
| | cdk1, apoptosis, | | |
| | membrane fusion, | | |
| | microtubule bundling, | | |
| | phosphotransferase | | |
| | activity, nuclear RNA | | |
| | export, programmed | | |
| | neuronal cell death, | | |
| | DNA replication, and | | |
| | DNA repair. Found on | | |
| | chromosome 12 location | | |
| | 12p13. | | |
| CD4 | A type I transmembrane | Yes, envelope | Interacts with specific |
| | protein found on | | domains of gp120 |
| | helper/inducer T cells, | | facilitating viral fusion. |
| | monocytes, | | |
| | macrophages, and | | |
| | dendritic cells that is | | |
| | involved in T-cell | | |
| | recognition of antigens. | | |
| | Found on chromosome | | |
| | 12 location 12pter-p12. | | |
| CXCR4 | Binds chemokine SDF-1 | Yes, envelope | Viral co-receptor |
| | (stromal cell derived | | determines viral |
| | factor 1). Found on | | tropism for CD4 T |
| | hematopoietic | | cells. |
| | precursors, mature | | |
| | white blood cells and | | |
| | plasma cells. Found on | | |
| | chromosome 2 location | | |
| | 2q21. Type III | | |
| | transmembrane protein | | |
| | crossing the plasma | | |
| | membrane seven times. | | |
| CCR5 | Found on Th1 cells, | Yes, envelope | Viral co-receptor |
| | dendritic cells, | | determines viral |
| | monocytes/macrophages. | | tropism for |
| | Type III | | macrophages. |
| | transmembrane protein | | |
| | crossing the plasma | | |
| | membrane seven times. | | |
| | Ligands include | | |
| | monocyte chemo | | |
| | attractant protein 2 | | |
| | (MCP-2), macrophage | | |
| | inflammatory protein 1 | | |
| | alpha (MIP-1 alpha), | | |
| | macrophage | | |
| | inflammatory protein 1 | | |
| | beta (MIP-1 beta) and | | |
| | regulated on activation | | |
| | normal T expressed and | | |
| | secreted protein | | |
| | (RANTES). Found on | | |
| | chromosome 3 location | | |
| | 3p21.31 | | |
| NF_{K}B | Cellular transcription | | Binding sites in the |
| | factor involved in the | | viral LTR necessary |
| | immune process. | | for viral transcription. |
| | Found on chromosome * | | |
| | location *. | | |
| NFAT | Cellular transcription | | Binding sites in the |
| | factor involved in the | | viral LTR necessary |
| | immune process. | | for viral transcription. |
| | Found on chromosome | | |
| | 20 location 20q13.2-q13.3. | | |
| Sp1 | Cellular transcription | | Binding sites in the |
| | factor involved in the | | viral LTR necessary |
| | immune process. | | for viral transcription. |
| | Found on chromosome | | |
| | 12 location 12q13.1. | | |

What is claimed is:

1. A method for the production of a live animal model for HIV comprising the steps of:
a. creating and administering to said live animal a first set of HIV related human host proteins necessary for HIV to attach, penetrate, and replicate within said live animal using recombinant technology to encode said first set of proteins into commensal organisms normally found in and derived from gut associated lymphoid tissue of said animal;
b. creating and administering to said live animal, a second set of HIV related proteins necessary for HIV to evade said animal's immune response using recombinant technology to encode said second set of HIV related human host proteins into commensal organisms normally found in and derived from gut associated lymphoid tissue of said animal; and
c. infecting said animal with live, replication competent HIV.

2. The method of claim 1, wherein said first set and said second set of proteins are administered in trans and mirror concentrations found in normal human immunologic milieu.

3. The method of claim 1, wherein said method further comprises the step of coupling said first set and said second set of proteins with cell penetrating peptides using recombinant technology.

4. The method of claim 1, wherein said method further comprises the step of administering CypA-binding drug Cyclosporine to said live animal.

5. The method of claim 1, wherein said method further comprises the step of administering soluble complement-receptor 1 to said live animal.

6. The method of claim 1, wherein said method further comprises the step of administering Tat protein to said live animal.

7. The method of claim 1, wherein the first set of proteins comprise transcription factors, cellular cofactors, cellular receptors, cellular co-receptors, cellular proteases, cellular proteins involved in the ubiquitin-proteasome pathway, cellular adaptor proteins, and human ribosomal RNA.

8. The method of claim 7, wherein the transcription factors are selected from the group consisting of NF_{K} B, NFAT, Sp1, and combinations thereof.

9. The method of claim 7, wherein the cellular cofactors are selected from the group consisting of Cyclin T, CDK9/PITALRE, RNA polymerase II, Exportin 1/Crm 1, Ran GTP, Ran GTPase activating protein (RanGAP), Ran Binding Protein (RanBP1), and combinations thereof.

10. The method of claim 7, wherein the cellular receptors are CD4.

11. The method of claim 7, wherein the cellular coreceptors are selected from the group consisting of CCR5, CXCR4, CCR2B, CCR3, CCR8, GPR1, GPR15 (Bob), STRL33 (Bonzo), US28, CX3CR1 (V28), APJ, chemR23, and combinations thereof.

12. The method of claim 7, wherein the cellular proteases are Furin.

13. The method of claim 7, wherein the cellular proteins involved in the ubiquitin-proteosome pathway are selected from the group consisting of H-β-TrCP, Skp1p, and combinations thereof.

14. The method of claim 7, wherein the cellular adaptor proteins are AP-2.

15. The method of claim 1, wherein the second set of proteins comprise plasma proteins, cell membrane bound proteins, and homologous restriction factor (HRF).

16. The method of claim 15, wherein the plasma proteins are selected from the group consisting of C4 binding protein (C4b protein), factor H and combinations thereof.

17. The method of claim 15, wherein the cell membrane bound proteins are selected from the group consisting of membrane cofactor protein (MCP), CD46, decay accelerating factor (CD55), complement-receptor 1 (CD35), complement-receptor 2 (CD21), homologous restriction factor, and combinations thereof.

18. The method of claim 1, wherein corresponding to HIV-1 said first set of proteins comprise Thy-1 (CD90), GM1 (β-galactosidase), HLA-DR, VCAM-1, VLA-4, MHC-1, CD63, CD81, CD82, CD107a, HP68, ezrin, moesin, cofilin, actin, ubiquitin, Pin1, tRNA synthetase, aminoacyl tRNA synthetase, GAPDH, MAPK/ERK2, HSP60, HSP70, HSC70, CypA, FKBP12, Tsg101, Tal, VPS28, AIP1/ALIX, VPS4B, UNG, Staufen, INI1, EF-1α, LEDGF/p75, PSIP2, DNA-PK, Ku80, hRad18, EED, HMGA/HMG-1a, BAF/BANF1, p300, Rev cofactor, HSp90, CypB, HSP 27, HSP40, VPS37B, CD4, CXCR4, CCR5, CD86, Phosphatidyl inositol 4,5-bisphosphate, NF_{K} B, NFAT, Sp1, Cyclin T, CDK9/PITALRE, RNA polymerase II, Exportin 1/Crm 1, Ran GTP, Ran GTPase activating protein, Ran Binding Protein, CCR2B, CCR3, CCR8, GPR1, GPR15, STRL33, US28, CX3CR1, APJ, chemR23, Furin, AP-2, CD35, CD21 and tRNA^{lys}; and wherein corresponding to HIV-1 said second set of proteins comprise MCP/CD46, DAF/CD55, HRF-20/CD59, Factor H, HLA-DR, ICAM-1, ICAM-2, ICAM-3, LFA-1, VCAM-1, MHC-1, CD63, CD81, CD82, CD107a, ubiquitin, CypA, Tsg101, Tal, VPS28, AIP1/ALIX APOBEC3G, APOBEC3F, HSp90, CD4, CXCR4, CCR5, CD86, CCR2B, CCR3, CCR8, GPR1, GPR15, STRL33, US28, CX3CR1, APJ, H-β-TrCP, Skp1p, C4 binding protein, CD35, and CD21.

19. The method of claim 1, wherein corresponding to HIV-2 said first Set of proteins comprise HLA-DR, MHC-1, HSP70, UNG, Staufen, α-actinin 1, LEDGF/P75, tRNA synthetase, aminoacyl tRNA synthetase, tRNA^{lys}, GAPDH, CD4, CXCR4, CCR5, NF_{K}B, NFAT, and Sp1, and wherein corresponding to HIV-2 said second set of proteins comprise HLA-DR, MHC-1, CD4, CXCR4, and CCR5.

20. A composition comprising:
a. a first set of HIV related human host proteins necessary for a HIV virion to attach, penetrate, and replicate within a live animal, wherein said proteins are encoded in a genetically engineered commensal organism normally found in and derived from gut associated lymphoid tissue of said live animal using recombinant technology;
b. a second set of HIV related human host proteins necessary for HIV to evade said animal's immune response, wherein said proteins are encoded in a genetically engineered commensal organism normally found in and derived from gut associated lymphoid tissue of said live animal using recombinant technology; and
c. live, replication competent HIV.

21. The composition of claim 20, wherein said first set and said second set of proteins are supplied in trans and mirror concentrations found in the normal human immunologic milieu.

22. The composition of claim 20, wherein said first set and said second set of proteins are coupled with DNA encoding a cell penetrating peptide using recombinant technology.

23. The composition of claim 20, in combination with CypA-binding drug Cyclosporine.

24. The composition of claim 20, in combination with soluble complement-receptor 1.

25. The composition of claim 20, in combination with Tat protein.

26. The composition of claim 20, wherein said first set of proteins comprise transcription factors, cellular cofactors, cellular receptors, cellular co-receptors, cellular proteases, cellular proteins involved in the ubiquitin-proteasome pathway, cellular adaptor proteins, human ribosomal RNA, and combinations thereof.

27. The composition of claim 26, wherein the transcription factors are selected from the group consisting of NF_{K}B, NFAT, Sp1, and combinations thereof.

28. The composition of claim 26, wherein the cellular cofactors are selected from the group consisting of Cyclin T, CDK9/PITALRE, RNA polymerase II, Exportin 1/Crm1, Ran GTP, Ran GTPase activating protein (RanGAP), Ran Binding Protein (RanBP1), and combinations thereof.

29. The composition of claim 26, wherein the cellular receptors are CD4.

30. The composition of claim 26, wherein the cellular coreceptors are selected from the group consisting of CCR5, CXCR4, CCR2B, CCR3, CCR8, GPR1, GPR15 (Bob), STRL33 (Bono), US28, CX3CR1 (V28), APJ, chemR23, and combinations thereof.

31. The composition of claim 26, wherein the cellular proteases are Furin.

32. The composition of claim 26, wherein the cellular proteins involved in the ubiquitin-proteasome pathway are selected from the group consisting of H-β-TrCP, Skp1p, and combinations thereof.

33. The composition of claim 26, wherein the cellular adaptor proteins are AP-2.

34. The composition of claim 20, wherein said second set of proteins comprise plasma proteins, cell membrane bound proteins, and homologous restriction factor (HRF).

35. The composition of claim 34, wherein the plasma proteins are selected from the group consisting of C4 binding protein (C4b protein), factor H, and combinations thereof.

36. The composition of claim 34, wherein the cell membrane bound proteins are selected from the group consisting of membrane cofactor protein (MCP), CD46, decay accelerating factor (CD55), complement-receptor 1 (CD35), complement-receptor 2 (CD21), homologous restriction factor, and combinations thereof.

37. The composition of claim 20, wherein corresponding to HIV-1 said first set of proteins comprise Thy-1 (CD90), GM1 (β-galactosidase), HLA-DR, VCAM-1, VLA-4, MHC-1, CD63, CD81, CD82, CD107a, HP68, ezrin, moesin, cofilin, actin, ubiquitin, Pin1, tRNA synthetase, aminoacyl tRNA synthetase, GAPDH, MAPK/ERK2, HSP60, HSP70, HSC70, CypA, FKBP12, Tsg101, Tal, VPS28, AIP1/ALIX, VPS4B, UNG, Staufen, INI1, EF-1α, LEDGF/p75, PSIP2, DNA-PK, Ku80, hRad18, EED, HMGA/HMG-1α, BAF/BANF1, p300, Rev cofactor, HSp90, CypB, HSP 27, HSP40, VPS37B, CD4, CXCR4, CCR5, CD86, Phosphatidyl inositol 4,5-bisphosphate, NF_{K}B, NFAT, Sp1, Cyclin T, CDK9/PITALRE, RNA polymerase II, Exportin 1/Crm 1, Ran GTP, Ran GTPase activating protein, Ran Binding Protein, CCR2B, CCR3, CCR8, GPR1, GPR15, STRL33, US28, CX3CR1, APJ, chemR23, Furin, AP-2, CD35, CD21 and tRNA^{lys}; and wherein corresponding to HIV-1 said second set of proteins comprise MCP/CD46, DAF/CD55, HRF-20/CD59, Factor H, HLA-DR, ICAM-1, ICAM-2, ICAM-3, LFA-1, VCAM-1, MHC-1, CD63, CD81, CD82, CD107a, ubiquitin, CypA, Tsg101, Tal, VPS28, AIP1/ALIX APOBEC3G, APOBEC3F, HSp90, CD4, CXCR4, CCR5, CD86, CCR2B, CCR3, CCR8, GPR1, GPR15, STRL33, US28, CX3CR1, APJ, H-β-TrCP, Skp1p, C4 binding protein, CD35, and CD21.

38. The composition of claim 20, wherein corresponding to HIV-2 said first set of proteins comprise HLA-DR, MHC-1, HSP70, UNG, Staufen, α-actinin 1, LEDGF/P75, tRNA synthetase, aminoacyl tRNA synthetase, tRNA^{lys}, GAPDH, CD4, CXCR4, CCR5, NF_{K}B, NFAT, and Sp1, and wherein corresponding to HIV-2 said second set of proteins comprise HLA-DR, MHC-1, CD4, CXCR4, and CCR5.
